(19) United States
(12) Patent Application Publication
(10) Pub. No.: US 2025/0195880 A1
(43) Pub. Date: Jun. 19, 2025

(54) IMPLANTABLE STIMULATING ASSEMBLY WITH MUSCLE

(71) Applicant: COCHLEAR LIMITED, Macquarie University, NSW (AU)

(72) Inventor: Charles Roger Aaron LEIGH, Macquarie University (AU)

(21) Appl. No.: 18/847,404

(22) PCT Filed: Mar. 17, 2023
(86) PCT No.: PCT/IB2023/052657

§ 371 Date: Sep. 16, 2024

Related U.S. Application Data
(60) Provisional application No. 63/320,903, filed on Mar. 17, 2022.

(51) Int. Cl. A61N 1/05; A61N 1/36

(52) U.S. Cl. CPC A61N 1/0541; A61N 1/36038

(57) ABSTRACT

An electrode array, such as a cochlear implant electrode array, or a retinal prosthesis array, or a vestibular array, comprising a plurality of electrodes and an electrode carrier carrying the plurality of electrodes. The electrodes can be made of platinum, for example, and the carrier can be made of silicone, for example. The electrode carrier includes an artificial muscle component.

### CROSS-REFERENCE TO RELATED APPLICATIONS

This application claims priority to U.S. Provisional Application No. 63/320,903, entitled IMPLANTABLE STIMULATING ASSEMBLY WITH MUSCLE, filed on Mar. 17, 2022, naming Charles Roger Aaron LEIGH as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an exemplary embodiment, there is an electrode array, comprising a plurality of electrodes and an electrode carrier carrying the plurality of electrodes, wherein the electrode carrier includes an artificial muscle component.

In an exemplary embodiment, there is a method, comprising obtaining an implantable electrode array, inserting the implantable electrode array into a recipient and during insertion and/or subsequent to the full insertion of the implantable component, controllably transforming the electrode array from a first geometry to a second geometry without any of: external force relief controlling the transformation, external pressure relief controlling the transformation and reaction force controlling the transformation.

In an exemplary embodiment, there is an implantable apparatus, comprising an electrode array including a plurality of electrodes and a carrier carrying electrodes, wherein the electrode array is configured so that, during and/or after insertion into a recipient, a local radius of curvatures at a first location and a second location more distal than the first location can be simultaneously controllably changed relative to one another.

In an exemplary embodiment, there is an electrode array, comprising a plurality of implantable electrodes extending in an evenly spaced apparat manner, the electrodes being made of a precious metal and respectively connected to respective electrical leads and a flexible silicone electrode carrier body carrying the plurality of electrodes, wherein the electrode carrier has a length that is at least five times a maximum thickness thereof, the electrode leads extend inside the electrode carrier, and the electrode carrier includes at least one hydrogel body located in a cutout of the carrier, the hydrogel body being an artificial muscle.

### BRIEF DESCRIPTION OF THE DRAWINGS

Embodiments of the present invention are described below with reference to the attached drawings, in which:

FIG. 1A is a perspective view of an exemplary hearing prosthesis utilized in some exemplary embodiments;

FIG. 1B is a side view of the implantable components of the cochlear implant illustrated in FIG. 1A;

FIG. 2 is a side view of an embodiment of the electrode array illustrated in FIGS. 1A and 1B in a curled orientation;

FIG. 3 is an exemplary schematic of a retinal prosthesis;

FIG. 4 is an exemplary schematic of a vestibular array;

FIG. 5 is a functional schematic of an electrode array including 22 electrodes spaced apart from one another;

FIG. 6 is an exemplary electrode array according to an exemplary embodiment;

FIG. 6A-7 are exemplary schematics of an actuator according to an exemplary embodiment;

FIGS. 8-10 and 11-19 are examples of various electrode arrays according to some embodiments, and in some instances, a positional shape thereof;

FIG. 20 is an exemplary flowchart for an exemplary method;

FIGS. 21 and 22 are exemplary electrode arrays according to some embodiments; and

FIGS. 23 and 10A show exemplary longitudinal axes according to some embodiments with associated details.

### DETAILED DESCRIPTION

Merely for ease of description, the techniques presented herein are described herein with reference by way of background to an illustrative medical device, namely a cochlear implant. However, it is to be appreciated that the techniques presented herein may also be used with a variety of other medical devices that, while providing a wide range of therapeutic benefits to recipients, patients, or other users, may benefit from setting changes based on the location of the medical device. For example, the techniques presented herein may be used to determine the viability of various types of prostheses, such as, for example, a vestibular implant and/or a retinal implant, with respect to a particular human being. And with regard to the latter, the techniques presented herein are also described with reference by way of background to another illustrative medical device, namely a retinal implant. The techniques presented herein are also applicable to the technology of vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc.

Also, embodiments are directed to other types of hearing prostheses, such as middle ear implants, bone conduction devices (active transcutaneous, passive transcutaneous, percutaneous), and conventional hearing aids. Thus, embodiments are directed to devices that include implantable portions and embodiments that do not include implantable portions.

Any reference to one of the above-noted sensory prostheses corresponds to an alternate disclosure using one of the other above-noted sensory prostheses unless otherwise noted providing that the art enables such.

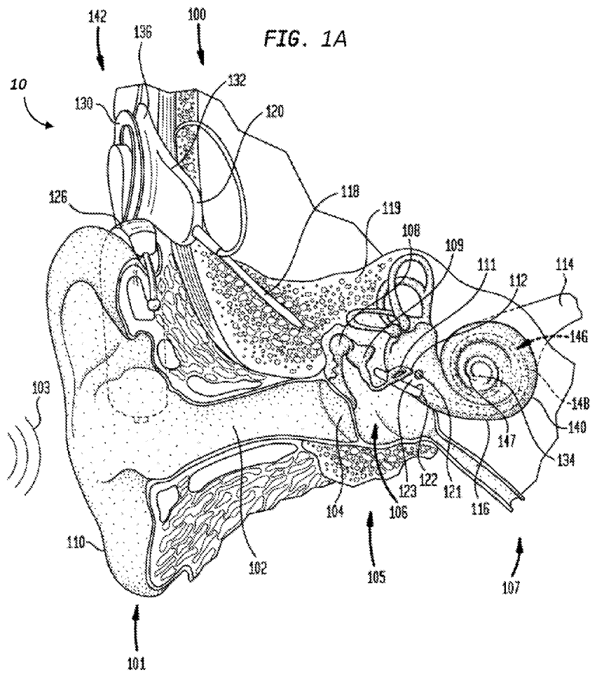

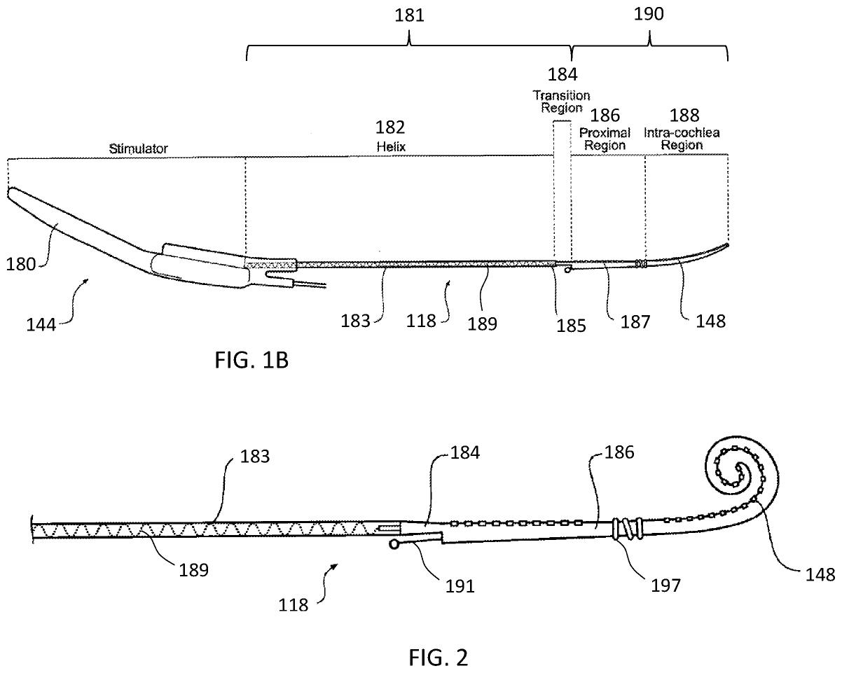

FIG. 1A is a perspective view of a totally implantable cochlear implant according to an exemplary embodiment, referred to as cochlear implant 100, implanted in a recipient. The cochlear implant 100 is part of a system 10 that can include external components, as will be detailed below.

In an alternate embodiment, the cochlear implant system is not a totally implantable system. By way of example, the cochlear implant system includes an external component that includes a microphone and a sound processor. The sound processor processes signals from the microphone, and generates a signal that is transmitted transcutaneously to an implantable component which then uses the signal to stimulate tissue and evoke a hearing percept.

It is noted that in some conventional parlances, the entire system 10 is referred to as a cochlear implant, especially in the case of a cochlear implant that is not totally implantable. Herein, the phrase cochlear implant refers to the implantable component, and the phrase cochlear implant system refers to the entire system 10. That is, the phrase cochlear implant corresponds to the implantable component of a non-totally implantable cochlear implant system.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1A with an external device 142, that is part of system 10 (along with cochlear implant 100), which, as described below, is configured to provide power to the cochlear implant.

In the illustrative arrangement of FIG. 1A, external device 142 may comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1A, the external energy transfer assembly comprises an external coil 130 that forms part of an inductive radio frequency (RF) communication link. External coil 130 is typically a wire antenna coil comprised of multiple turns of electrically insulated single-strand/or multi-strand platinum or gold wire. External device 142 also includes a magnet (not shown) positioned within the turns of wire of external coil 130. It should be appreciated that the external device shown in FIG. 1A is merely illustrative, and other external devices may be used with embodiments of the present invention.

Cochlear implant 100 comprises an internal energy transfer assembly 132 which may be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand/or multi-strand platinum or gold wire.

Cochlear implant 100 further comprises a main implantable component 120 and an elongate stimulating assembly 118. In embodiments of the present invention, internal energy transfer assembly 132 and main implantable component 120 are hermetically sealed within a biocompatible housing. In embodiments of the present invention, main implantable component 120 includes a sound processing unit (not shown) to convert the sound signals received by the implantable microphone in internal energy transfer assembly 132 to data signals. Main implantable component 120 further includes a stimulator unit (also not shown) which generates electrical stimulation signals based on the data signals. The electrical stimulation signals are delivered to the recipient via elongate stimulating assembly 118.

Elongate stimulating assembly 118 has a proximal end connected to main implantable component 120, and a distal end implanted in cochlea 140. Stimulating assembly 118 extends from main implantable component 120 to cochlea 140 through mastoid bone 119. In some embodiments stimulating assembly 118 may be implanted at least in basal region 116, and sometimes further. For example, stimulating assembly 118 may extend towards apical end of cochlea 140, referred to as cochlea apex 134. In certain circumstances, stimulating assembly 118 may be inserted into cochlea 140 via a cochleostomy 122. In other circumstances, a cochleostomy may be formed through round window 121, oval window 112, the promontory 123, or through an apical turn 147 of cochlea 140.

Stimulating assembly 118 comprises a longitudinally aligned and distally extending array 146 of electrodes 148, disposed along a length thereof. As noted, a stimulator unit generates stimulation signals which are applied by stimulating contacts 148, which in an exemplary embodiment are electrodes, to cochlea 140, thereby stimulating auditory nerve 114. In an exemplary embodiment, stimulation contacts can be any type of component that stimulates the cochlea (e.g., mechanical components, such as piezoelectric devices that move or vibrate, thus stimulating the cochlea (e.g., by inducing movement of the fluid in the cochlea), electrodes that apply current to the cochlea, etc.). Embodiments detailed herein will generally be described in terms of a stimulating assembly 118 utilizing electrodes as elements 148. It is noted that alternate embodiments can utilize other types of stimulating devices. Any device, system, or method of stimulating the cochlea can be utilized in at least some embodiments.

As noted, cochlear implant 100 comprises a totally implantable prosthesis that is capable of operating, at least for a period of time, without the need for external device 142. Therefore, cochlear implant 100 further comprises a rechargeable power source (not shown) that stores power received from external device 142. The power source may comprise, for example, a rechargeable battery. During operation of cochlear implant 100, the power stored by the power source is distributed to the various other implanted components as needed. The power source may be located in main implantable component 120, or disposed in a separate implanted location.

It is noted that the teachings detailed herein and/or variations thereof can be utilized with a non-totally implantable prosthesis. That is, in an alternate embodiment of the cochlear implant 100, the cochlear implant 100, and thus system 10, is a traditional hearing prosthesis.

While various aspects of the present invention are described with reference to a cochlear implant (whether it be a device utilizing electrodes or stimulating contacts that impart vibration and/or mechanical fluid movement within the cochlea), it will be understood that various aspects of the embodiments detailed herein are equally applicable to other stimulating medical devices having an array of electrical simulating electrodes such as auditory brain implant (ABI), functional electrical stimulation (FES), spinal cord stimulation (SCS), penetrating ABI electrodes (PABI), and so on. Also, while embodiments disclosed herein are directed to electrodes, it is noted that in other embodiments, the teachings detailed herein are applicable to non-electrical stimulation, such as by way of example only and not by way of limitation, optical stimulation, magnetic stimulation, etc. Indeed, in an exemplary embodiment, instead of or in addition to electrodes, induction coils are utilized to stimulate the tissue (e.g., the tissue inside the cochlea). Moreover, it is noted that embodiments disclosed herein are not limited to application to hearing prostheses. For example, the teachings detailed herein can be applicable to retinal stimulation, skin stimulation, etc. Note further that the teachings detailed herein are applicable to deep brain stimulation, and thus an exemplary embodiment includes a deep brain stimulator assembly utilizing the teachings detailed herein. Further, it is noted that the teachings herein are applicable to stimulating medical devices having electrical stimulating electrodes of all types such as straight electrodes, perimodiolar electrodes and short/basal electrodes. Also, various aspects of the embodiments detailed herein and/or variations thereof are applicable to devices that are non-stimulating and/or have functionality different from stimulating tissue, such as for example, any intra-body dynamic phenomenon (e.g., pressure, or other phenomenon consistent with the teachings detailed herein) measurement/sensing, etc., which can include use of these teachings to sense or otherwise detect a phenomenon at a location other than the cochlea (e.g., within a cavity containing the brain, the heart, etc.). Additional embodiments are applicable to bone conduction devices, Direct Acoustic Cochlear Stimulators/Middle Ear Prostheses, and conventional acoustic hearing aids. Any device, system, or method of evoking a hearing percept can be used in conjunction with the teachings detailed herein. The teachings detailed herein are applicable to any device, system, or method where an elongate lead having elastic properties or the like has utilitarian value with respect to positioning thereof.

Still focusing on a cochlear implant, FIG. 1B is a side view of the cochlear implant 100 without the other components of system 10 (e.g., the external components). Cochlear implant 100 comprises a receiver/stimulator 180 (combination of main implantable component 120 and internal energy transfer assembly 132) and an elongate stimulating assembly 118. Stimulating assembly 118 includes a helix region 182 that includes a body 183 in which is embedded (e.g., in the case where the body is silicone or another biocompatible material molded around wire leads) or otherwise containing (e.g., in the case where the body is a conduit or tube) electrical lead wires 189 in a helix (more on this below), a transition region 184 (which can be part of the body 183), a proximal region 186, and an intra-cochlear region 188. The proximal region 186, in this embodiment, is connected to the transition region 184 via a distinct connection 185, although in other embodiments, the transition region is blended into the helix region 182 (and the proximal region 186). Proximal region 186 and intra-cochlear region 188 form an electrode array 190. The portion of the stimulating assembly 118 that extends from the receiver/stimulator 180 to the electrode array 190 is referred to herein as the lead assembly, indicated by reference numeral 181 in FIG. 1A. In an exemplary embodiment, proximal region 186 is located in the middle-ear cavity of the recipient after implantation of the intra-cochlear region 188 into the cochlea. Thus, proximal region 186 corresponds to a middle-ear cavity sub-section of the stimulating assembly 118. In some exemplary embodiments, nubs 187 are provided on the outer surface of the proximal region to aid in the manipulation of the electrode array assembly 190 during insertion of the intra-cochlear region into the cochlea. Electrode array assembly 190, and in particular, intra-cochlear region 188 of electrode array assembly 190, supports a plurality of electrode contacts 148. These electrode contacts 148 are each connected to a respective conductive pathway, such as wires, PCB traces, etc. (not shown) which are connected to receiver/stimulator 180, through which respective stimulating electrical signals for each electrode contact 148 travel.

It is noted that in some embodiments, the helix region 182 does not extend as far as that depicted in FIG. 1A, and the transition region 184 is thus longer. That is, in some exemplary embodiments, the helix region 182 does not extend substantially the full length between the receiver/stimulator 180 and the proximal region 186, but instead extends less than that (e.g., about half the distance), where the remaining distance is established by substantially straight lead wires, or at least wires that are not substantially helixed. Any arrangement of lead wires that can enable the teachings detailed herein and/or variations thereof to be practiced can be utilized in some exemplary embodiments.

FIG. 2 is a side view of a portion of stimulating assembly 118 where the electrode array of the electrode array assembly 190 is in a curled orientation, as it would be when inserted in a recipient's cochlea, with electrode contacts 148 located on the inside of the curve.

It is noted that FIGS. 1B and 2 can be, by way of example only and not by way of limitation, a perimodiolar stimulating assembly or a mid-scala assembly which assumes a mid-scala position during or following implantation.

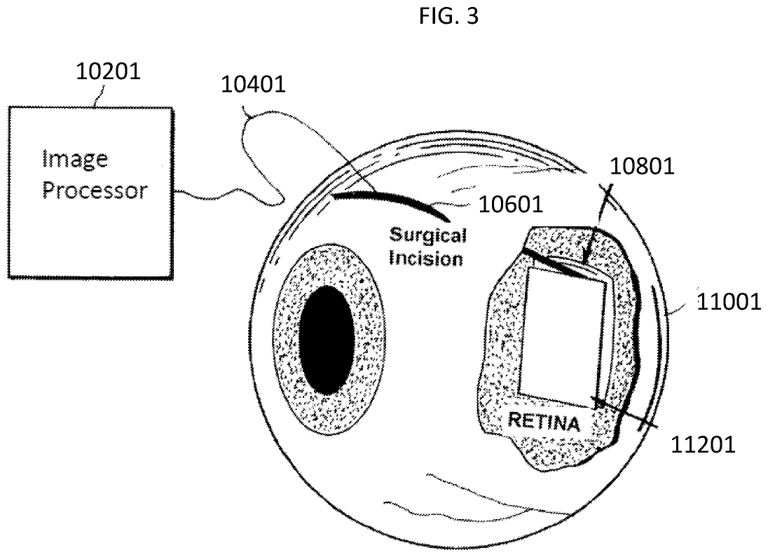

FIG. 3 presents an exemplary embodiment of a neural prosthesis in general, and a retinal prosthesis and an environment of use thereof, in particular, the components of which can be used in whole or in part, in some of the teachings herein. In some embodiments of a retinal prosthesis, a retinal prosthesis sensor-stimulator 10801 is positioned proximate the retina 11001. In an exemplary embodiment, photons entering the eye are absorbed by a microelectronic array of the sensor-stimulator 10801 that is hybridized to a glass piece 11201 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 10201 is in signal communication with the sensor-stimulator 10801 via cable 10401 which extends through surgical incision 00601 through the eye wall (although in other embodiments, the image processor 10201 is in wireless communication with the sensor-stimulator 10801). The image processor 10201 processes the input into the sensor-stimulator 10801 and provides control signals back to the sensor-stimulator 10801 so the device can provide processed output to the optic nerve. That said, in an alternate embodiment, the processing is executed by a component proximate with or integrated with the sensor-stimulator 10801. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 10801 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

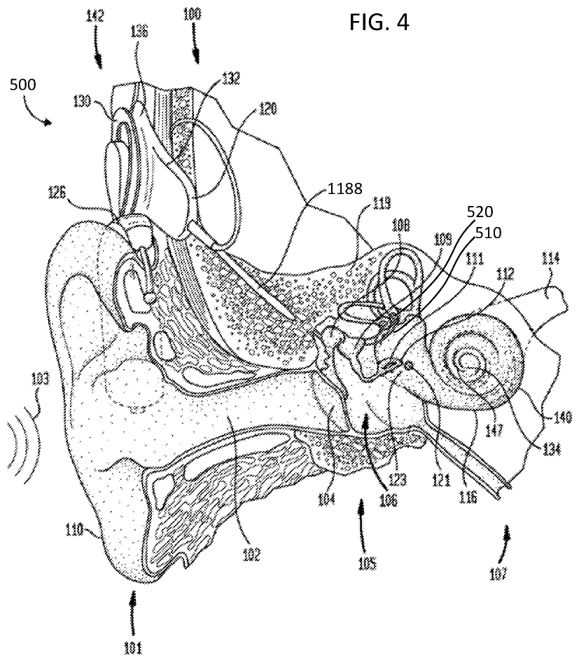

FIG. 4 depicts an exemplary vestibular implant 500 according to one example. Some specific features are described utilizing the above noted cochlear implant of FIG. 1 in contacts for the various elements. In this regard, some features of a cochlear implant are utilized with vestibular implants. In the interest of textual and pictorial economy, various elements of the vestibular implant that generally correspond to the elements of the cochlear implant above are referenced utilizing the same numerals. Still, it is noted that some features of the vestibular implant 500 will be different from that of the cochlear implant above. By way of example only and not by way of limitation, there may not be a microphone on the behind-the-ear device 126. Alternatively, sensors that have utilitarian value in the vestibular implant can be contained in the BTE device 126. By way of example only and not by way of limitation, motion sensors can be located in BTE device 126. There also may not be a sound processor in the BTE device. Conversely, other types of processors, such as those that process data obtained from the sensors, will be present in the BTE device 126. Power sources, such as a battery, will also be included in the BTE device 126. Consistent with the BTE device of the cochlear implant of FIG. 1, a transmitter/transceiver will be located in the BTE device or otherwise in signal communication therewith.

The implantable component includes a receiver stimulator in a manner concomitant with the above cochlear implant. Here, the vestibular stimulator comprises a main implantable component 120 and an elongate electrode assembly 1188 (where the elongate electrode assembly 1188 has some different features from the elongate electrode assembly 118 of the cochlear implant, some of which will be described shortly). In some embodiments, internal energy transfer assembly 132 and main implantable component 120 are hermetically sealed within a biocompatible housing. In some embodiments, main implantable component 120 includes a processing unit (not shown) to convert data obtained by sensors, which could be on board sensors implanted in the recipient, into data signals.

Main implantable component 120 further includes a stimulator unit (also not shown) which generates electrical stimulation signals based on the data signals. The electrical stimulation signals are delivered to the recipient via elongate electrode assembly 1188.

It is briefly noted that while the embodiment shown in FIG. 4 represents a partially implantable vestibular implant, embodiments can include a totally implantable vestibular implant, such as, where, for example, the motion sensors are located in the implantable portion, in a manner analogous to a cochlear implant.

Elongate electrode assembly 1188 has a proximal end connected to main implantable component 120, and extends through a hole in the mastoid 119, in a manner analogous to the elongate electrode assembly 118 of the cochlear implant, and includes a distal end that extends to the inner ear. In some embodiments, the distal portion of the electrode assembly 1188 includes a plurality of leads 510 that branch out away from the main body of the electrode assembly 118 to electrodes 520. Electrodes 520 can be placed at the base of the semicircular ducts as shown in FIG. 4. In an exemplary embodiment, one or more of these electrodes are placed in the vicinity of the vestibular nerve branches innervating the semicircular canals. In some embodiments, the electrodes are located external to the inner ear, while in other embodiments, the electrodes are inserted into the inner ear. Note also while this embodiment does not include an electrode array located in the cochlea, in other embodiments, one or more electrodes are located in the cochlea in a manner analogous to that of a cochlear implant.

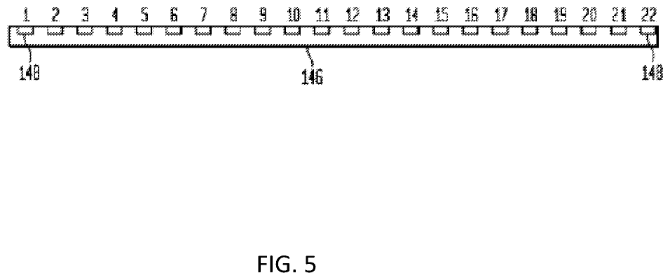

FIG. 5 illustrates a more detailed view, albeit functionally, of an exemplary electrode array 146 comprising a plurality of electrodes 148 labeled 1-22, in accordance with an embodiment. In an exemplary embodiment, each electrode 148 is an electrode that corresponds to a specific frequency band channel of the cochlear implant 100, where electrode 22 corresponds to the lowest frequency band (channel), and electrode 1 corresponds to the highest frequency band (channel). Briefly, it is noted that during stimulation by the electrodes to evoke a hearing percept, one or more electrodes 148 is activated at a given electrode stimulation level (e.g., current level).

In an exemplary embodiment, the electrode array assembly 190 includes at least an intra-cochlear region where the carrier of the electrodes 148 (the electrode carrier) is made of a viscoelastic material. In an exemplary embodiment, the carrier of the electrodes 148 is made of viscoelastic polyurethane foam, which in some embodiments can be a memory foam, a polyurethane with additional chemicals that increase the material's viscosity and density, a material such as what is utilized in earplugs, etc. In an exemplary embodiment, the electrode carrier is made of regular medical grade silicone, such as non-viscoelastic silicone, an example of which is Nusil's 48-series of medical grade liquid silicone rubber, and is not made of viscoelastic silicone.

By “made of,” it is meant that the component at issue is at least 50.1% by weight of the material at issue (not including impurities). In an exemplary embodiment, the component at issue is at least 60%, 70%, 80%, 90%, or 100% by weight constructed of the material at issue (not including impurities).

Some embodiments are directed to enabling cochlear implant electrode arrays to have different shapes at different times. Embodiments can enable the electrode array to be straight(ish) during insertion into the cochlea, and then follow the spiral of the cochlea beyond that which results from the mere elasticity of the array (although embodiments rely in part on the elasticity in some embodiments). Unlike some prior arrays, where the electrode array is floppy (including during insertion and/or after insertion) so as to conform to the cochlea as the array is pushed into the cochlea, or those that are precurved and use a mechanism (stylet and/or sheath) to hold the array straight when required, embodiments can enable the control of the shape of the array (shape with respect to the longitudinal axis—the curve of the array over the longitudinal direction). Moreover, some embodiments enable more precise control and/or enable adjustment of the overall curvature of the array. This as opposed to the mere concept where when the stylet is removed, the array curls as much as it can, constrained by the tissue, or simply curls until the elasticity reaches equilibrium, or the concept where the tissue itself controls the curvature/geometry of the array. In some embodiments, the teachings herein enable tweaking of the overall electrode array shape.

Some embodiments use miniature artificial muscles, where such can, in some embodiments, mimic the function of natural Animalia muscle. In some embodiments, hydrogel-based artificial muscles are utilized, at least if such possess the sufficient biocompatibility and suitability for miniaturization.

In some embodiments, the devices used to achieve the changes in shape of the array are polymer based with a preferred direction of the polymer chains. Polymer chains can be triggered to move between a shorter or longer phase (e.g., by turning on/off H-bonding). Triggers include changes in temperature, light, electricity, and/or pH, for example.

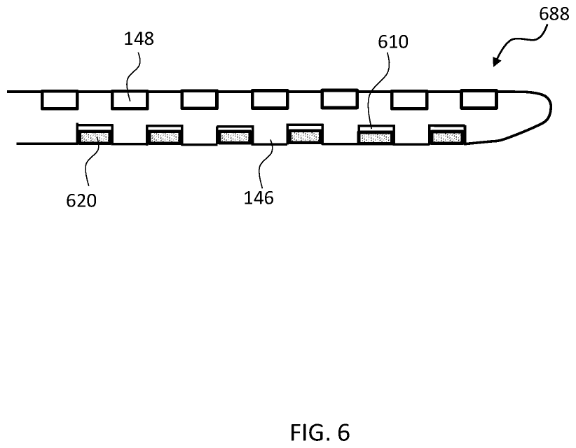

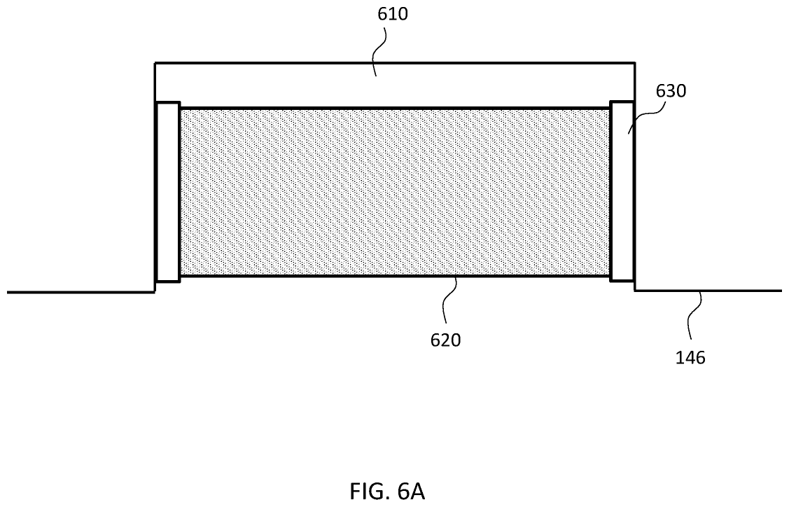

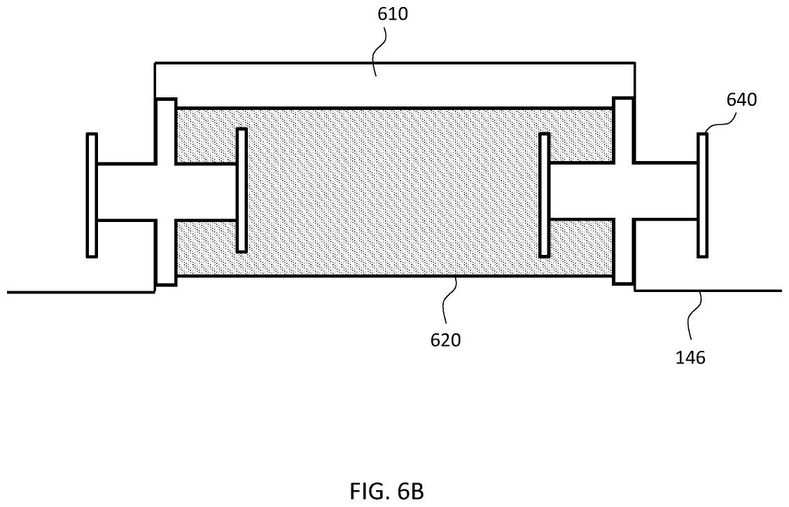

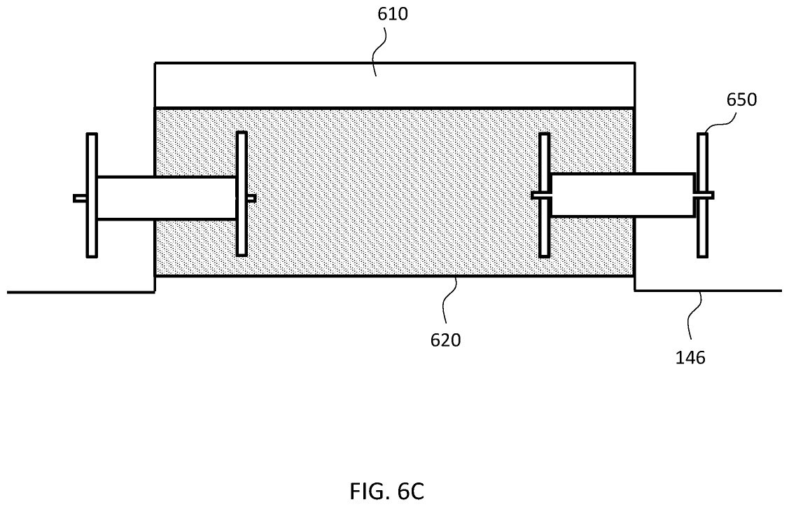

FIG. 6 shows a portion of an intra-cochlear portion 688 of a cochlear implant electrode array. As seen, unlike the conventional array, this array has cutouts 610 along the bottom of the array (the side opposite the electrodes 148) in which are located a series of hydromorphic polymer components 620 attached directly to the walls of the cutouts. In some embodiments, the attachment can be indirect, such as by plates 630 that are bonded on one side to the silicone body of the carrier and bonded on the opposite side to the components 620 as seen in FIG. 6A. Further, in an exemplary embodiment, attachment structure such as structure 640 is seen in FIG. 6B can be utilized, which structure has sub-plates attached to a main plate, which sub-plates are embedded in the silicone of the carrier 146 and the material of the polymer components 620 as shown. In an exemplary embodiment, the polymer components and/or the carrier body can be molded around the sub-plates. This can have utilitarian value in that the devices can be “locked” together owing to the interference fit established by the sub-plates.

FIG. 6C shows another exemplary embodiment where there is direct contact between the polymer components 620 and the silicone of the carrier body 146. Here, locking component's 650 are utilized to lock the polymer components 620 to the silicone body of the carrier member owing to the above noted interference fit.

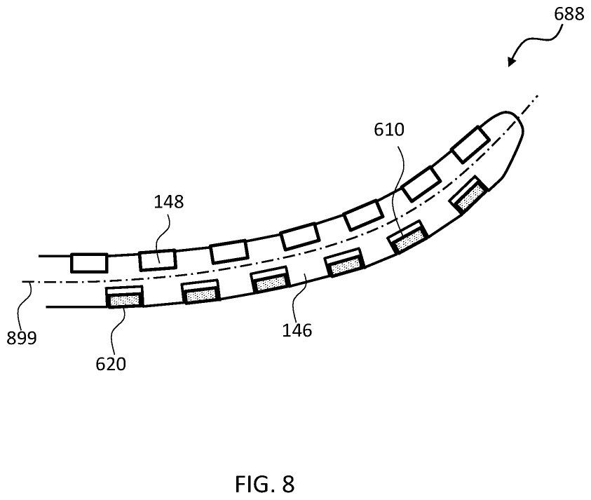

The array of FIG. 6 is a so-called pre-curved array, concomitant with the array of FIG. 2 above (but in the straightened state). That is, when the array is in a relaxed, unrestrained state, the array adopts a curved shape (as seen in FIG. 8), so as to better conform to the modiolus wall of the cochlea. Typically, in prior devices, a stiffening stylet is inserted/located in the array, to straighten the array, and as the array is advanced into the cochlea, the stylet is withdrawn, thus permitting the array to curl. Here, however, according to some embodiments, the polymer components are used to achieve the straightening and subsequent curving. But it is noted that embodiments herein can be utilized for an array that is straight in its relaxed state, where the polymer components “force” the array into a curved state. More on this below.

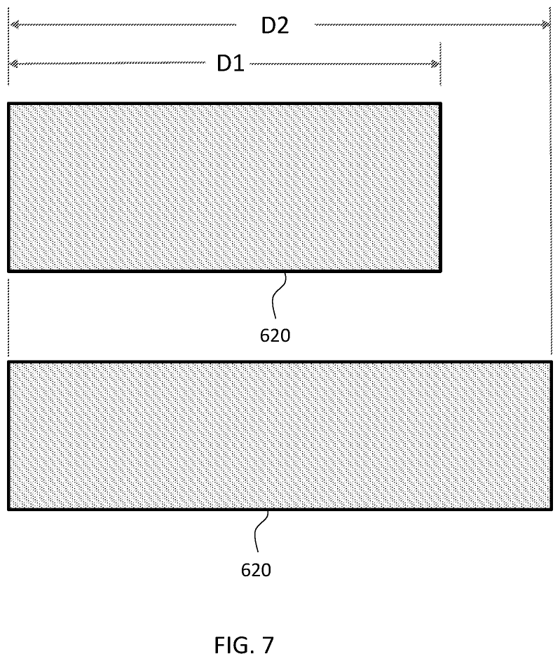

FIG. 6 shows that the polymer components 620 are distributed at even intervals along the section of the electrode array where a change of curvature over time is utilitarian, but it is noted that in some other embodiments, the distribution can be uneven. The polymer components can be actuated when a change of curvature is required by changing a variable that affects the component (e.g., the application of electricity, a change in temperature, etc.). FIG. 6 is an example of a curved electrode array that is “pulled” straight by shortening the polymer components 620 distributed along the length of the array. Thus, in this embodiment, the polymer components are artificial muscles (AMs), because they contract to do work. This as opposed to embodiments of the polymer components that expand to do work, which are not artificial muscles. FIG. 7 shows a comparison between a flex artificial muscle 620 having a length D1, and a relaxed artificial muscle 620 having a lengthy D2. The difference between D1 and D2 is the total amount that the artificial muscle is shortened when flexed from its relaxed state. As will be described in greater detail below, in some embodiments, the polymer component can be adjusted to have one or more different lengths between D1 and D2, at least for a temporary period of time, to obtain a radius of curvature between those which would exist for a polymer component having a length of D1 and D2 respectively. More on this below.

The embodiments above have the actuators and the cutouts therefore all located between the electrodes of the electrode array. Indeed, in the embodiments above, the beginnings and ends of the cutouts and the actuators are all located between the closest portions of adjacent electrodes with respect to location along the longitudinal axis of the electrode array. As will be seen below, in other embodiments, this is not necessarily the case. In an exemplary embodiment, of the actuators of the electrode array, at least 50, 55, 60, 65, 70, 75, 80, 85, 90, or 95% of the actuators and/or cutouts, or any value or range of values therebetween in 1% increments are located entirely in between the electrodes.

When the electrode array is straightened, this can, in some embodiments, facilitate insertion of the electrode array into the cochlea. After insertion of the electrode array into the cochlea, the artificial muscles could lengthen (relax/deactuate/be switched off) to allow the electrode to return to its curved shape, as seen in FIG. 8. In an alternate embodiment, to create a more gentle insertion, the tip AM could lengthen first and then progressively more proximal AMs could lengthen as the array progresses into the cochlea, as will be described in greater detail below. One way to achieve this is for the AMs to be “turned off”/relaxed as the electrode array is inserted in the cochlea. This can allow the array to adopt its perimodiolar shape. Actuators can be placed at locations that have curvature (with respect to the curved electrode array) to provide the “pulling” physical phenomenon that straightens the curve biased array (that is, in its relaxed state/if the actuators were not preset, the actuator would naturally curve). At locations with greater curvature (greater local radius of curvature with respect to the total array in its relaxed state), there can be more actuation (greater change in length of the actuator) at those locations to achieve straightening.

In an exemplary embodiment, the actuators when actuated and/or unactuated can provide stiffness to the electrode array. By way of example only and not by way of limitation, in the absence of a stylet for the like, a given cochlear implant electrode array is floppy or otherwise somewhat analogous to the tentacles of a squid. It is not a structure that has rigidity or otherwise is easy to insert into a cochlea for example. In an exemplary embodiment, the actuators can be such that upon actuation, a stiffness of the overall electrode array increases relative to that which would otherwise be the case. By way of example only and not by way limitation, in an exemplary embodiment, with respect to a section of the electrode array from the most proximal electrode to the most distal electrode, a force that would cause Euler buckling 1 mm or 2 or 3 mm from the unloaded state is is increased by at least 10, 15, 20, 25, 30, 35, 40, 45, 50, 55, 60, 65, 70, 75, 80, 90, 100, 125, 150, 175, 200, 250, 300, 350, 400, 450 or 500% or more or any value or range of values therebetween in 1% increments.

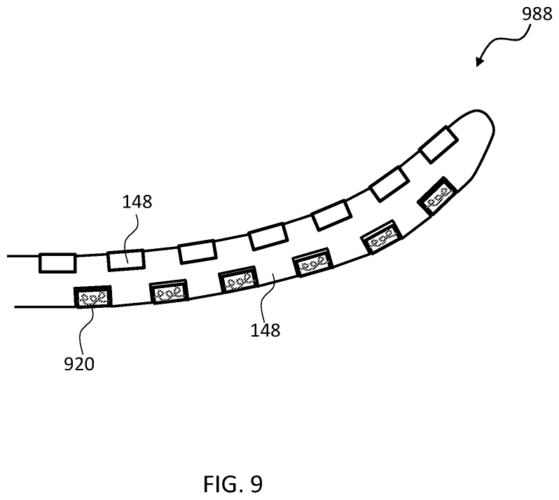

FIG. 9 shows another exemplary embodiment of an intracochlear portion 988 of a cochlear implant electrode array utilizing a different type of actuator 920 from the polymer component actuators detailed above. Here, actuators 920 are resistance actuators that heat and thus expand upon exposure to electricity. In an exemplary embodiment, the actuators can be MEMS actuators, such as flat plate actuators. The actuators can be any type of mechanical actuator that can enable the teachings detailed herein.

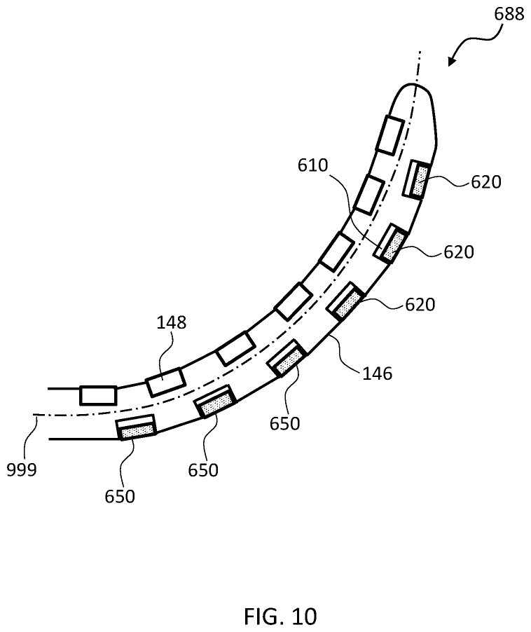

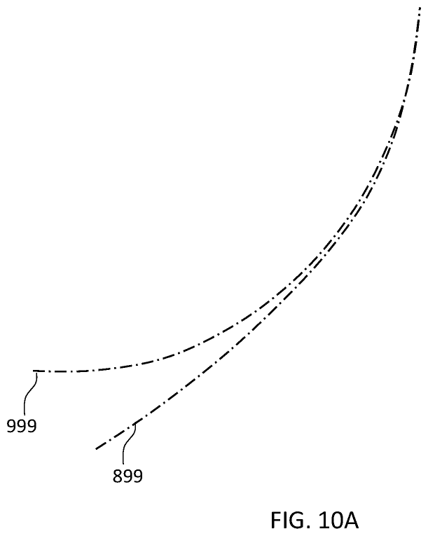

FIG. 8 depicts axis 899, which represents the longitudinal axis when the array is curved. In this embodiment, the radius of curvature of the longitudinal axis as shown in FIG. 8 is essentially the same along the length shown. This is because the actuators have been relaxed to the same lengths and the geometry of the array is such that when the actuators are relaxed, the curve of the axis is constant. In contrast, FIG. 10 depicts an example where the local radius of curvatures along the axis 999 changes along the length thereof. In this exemplary embodiment, this is because the relaxed state of the actuators 620 is less than the relaxed state of the actuators 650. Thus, the radius of curvature proximate the actuators 650 is greater than the radius of curvature proximate actuators 620. In this regard, the radius of curvature proximate the actuators 620 corresponds to that of FIG. 8. This can be seen in FIG. 10A where the two longitudinal axes are superimposed onto one another.

The local radius of curvatures can be different for reasons unrelated to the carrier. This as distinguished from what happens when a stylet is removed, where the shape is a result of the carrier.

In an alternative embodiment, the actuators can be the same, but the actuators are not all relaxed (at least fully relaxed). For example, actuators 650 are 75% relaxed, and actuators 620 are 100% relaxed, thus providing the difference in local radius of curvatures.

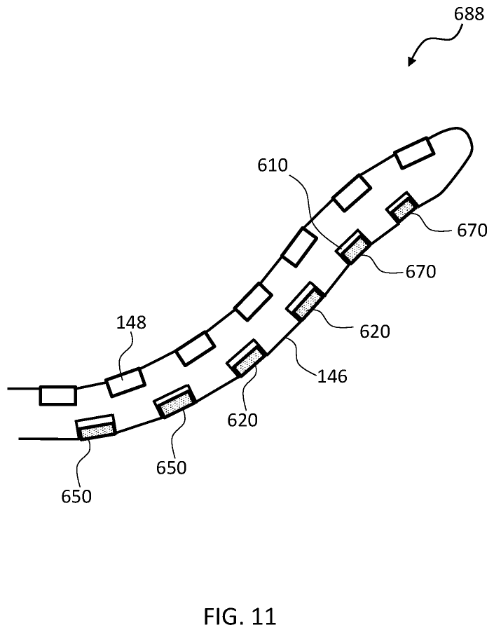

And note that in some embodiments, 100% contraction of the actuators is not needed or otherwise utilized to obtain the array in the straight configuration. In an exemplary embodiment, the actuators of FIG. 8 are actuators that are only 80% contracted. Thus, in some embodiments, the actuators could actually obtain a negative curvature of the electrode array by contracting more than 80% for example. This would be a slight negative curvature of the electrode array. And in this regard, FIG. 11 depicts an exemplary embodiment that utilizes different contraction states of the actuators to achieve different local radii of curvature. Here, the actuators labeled 620 have a contraction state concomitant on with the contraction state of FIG. 8. The actuators labeled 650 have a contraction state concomitant with the actuators labeled 650 in FIG. 10. The actuators labeled 670 have the greatest contraction state, which state is concomitant with the state of the actuators of FIG. 6 above. Thus, by variously controlling the contraction state of the actuators, different radii of curvatures and/or straight sections of the array can be achieved. This can be utilitarian with respect to snaking the electrode array into certain areas as the electrode array is advanced into those areas or otherwise through those areas. This can also have utilitarian value with respect to a custom fit where the embodiments described above have actuators that are in various states of contraction, array which can be controlled or otherwise developed or designed to have a unique ultimate shape after the array is fully implanted. And in this regard, it is noted that with respect to the embodiments above, where the actuators have been described as having different states of contraction and/or different states of relaxation to obtain the different local radius of curvatures and/or straight sections. It is noted that in alternate embodiment, different actuators of different designs can be utilized so that when the actuators are fully retracted or fully relaxed, the different radii of curvatures are obtained. This can have utilitarian value with respect to not having to continuously “power” or otherwise keep the actuators on to maintain that shape, which could be a complicating factor or otherwise a factor that increases design complexity with respect to long-term maintenance of that shape, which in the case of a cochlear implant electrode array, can be many years.

The embodiments described above have been mostly described in terms of a pre-curved array where the actuators, when actuated (at least fully contracted), hold the array straight. That is, the array is a molded array that is molded in a curved configuration (the relaxed state is curved), and the actuators restrain the array in the relaxed state. Embodiments can include the converse. The array is moulded straight and the actuators induce curvature when the actuators are actuated. In this embodiment, the actuators can be such that when the actuators are turned off, they expand, and thus induce the curvature. That said, in other embodiments, the actuators are such that the actuators have to be kept on to maintain the curvature. This could be a scenario where the actuators of FIG. 6 are utilized.

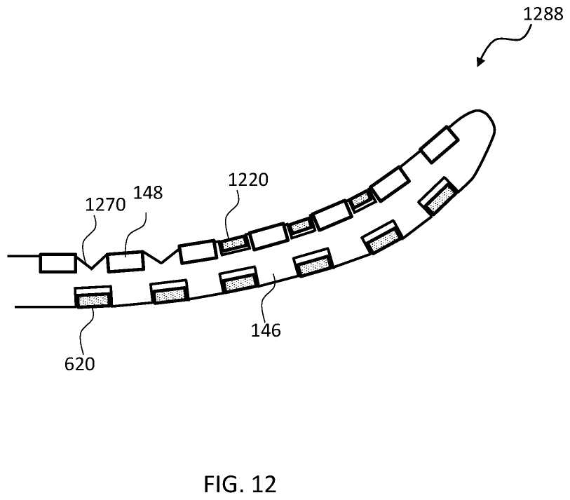

Embodiments also include geometries of the carrier body that further enhance or otherwise make movement (e.g., curling) of the carrier easier relative to that which would otherwise be the case. In this regard, FIG. 12 shows cutouts 1270 between electrodes on the perimodiolar side of the array. This provides for an area for the carrier body to collapse when the carrier body curves from the straight position. This can aid/improve the curlability of the array. (In an embodiment, hinges can be added between/at the actuators, while in other embodiments, the array is hingeless.) These cutouts can also be located on the other side of the electrode array as well (the lateral side of the array). Indeed, the areas 610 for the actuators 620 are in effect cutouts of the elastomer body that is the carrier member. Areas 610 happened to have a rectangular cross-section with respect to the views shown in FIG. 12, but would have a half-moon shaped cross-section with respect to a cross-section taken normal to the longitudinal axis. That half-moon shaped cross-section would also be the case for the triangular cutouts 1270 is taken on a plane normal to the longitudinal axis. Rectangular square cutouts can also be utilized on the perimodiolar side of the array.

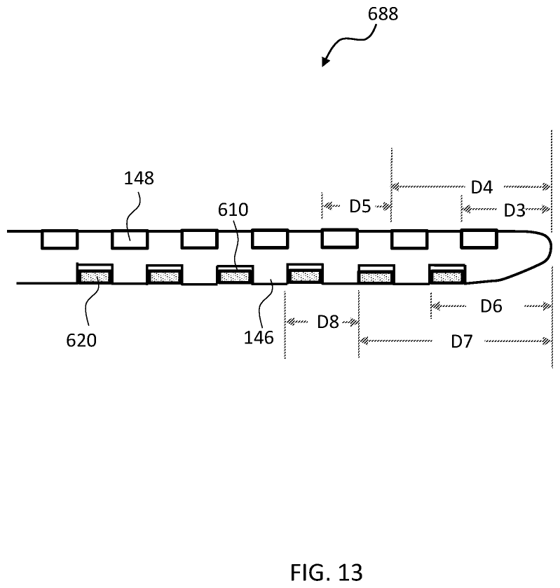

FIG. 13 depicts the electrode array in the straight configuration with dimensional lines thereon. The following reference values are for some exemplary embodiments and are not limiting. Other embodiments can have other values. In an exemplary embodiment, the distance D3, which is the distance from the most distal portion of the electrode array (the distal portion of the tip) to the portion of the most distal electrode 148 furthest from the tip can be less than or equal to or greater than 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm, or any value or range of values therebetween in 0.01 mm increments (e.g., 1.13 mm, 1.45 mm, 0.88 to 1.11 mm, etc.). D4, which is the distance from the tip to the portion of the second most distal electrode 148 furthest from the tip can be less than or equal to or greater than 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.25, 3.5, 3.75, 4, 4.25, 4.5, 4.75, 5, 5.25, 5.5, 5.75 or 6 mm, or any value or range of values therebetween in 0.01 mm increments (of course, D3 would not equal D4, and would not be values where there would be overlap the length of any given electrode, etc.—these values are simply values that can exist in some embodiments providing that the art enables such—and note that a length of an electrode can be less than or equal to and/or greater than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6. 1.65, 1.7, or 1.75 mm, or any value or range of values therebetween in 0.01 mm increments).

D5 which is the value from the most distal portion of the third electrode from the end to the most distal portion of the second electrode from the end can be any of the values of D3 (and need not be the same-indeed, D3 in practice will often be larger than D5). And note that the aforementioned value of D5 can be for a spacing of any electrode pair detailed herein with respect to the distal portions thereof, and thus the spacing of the distal most portions of electrodes 8 and 9 for example could be any of the values of D5, and the applicable value of D5 need not be the same as a D5 value for electrodes 5 and 6 for example. Again, the above values are simply exemplary numbers that can exist in at least some exemplary embodiments for any given permutation.

In an exemplary embodiment, the distance D6, which is the distance from the most distal portion of the electrode array (the distal portion of the tip) to the portion of the most distal cutout 610 furthest from the tip can be less than or equal to or greater than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.25, 3.5, 3.75, 4, 4.25, 4.5, 4.75, 5, 5.25, 5.5, 5.75, 6, 6.25, 6.5, 6.75 mm, or any value or range of values therebetween in 0.01 mm increments. And in this regard, it can be understood that in some embodiments, the distal most actuator 620 can be distal of the distal most electrode. That is, while the embodiments of the figures generally show actuators located, with respect to location along the longitudinal axis, between electrodes, in other embodiments, this is not necessarily the case. This can have utilitarian value with respect to steering the tip of the electrode array as the electrode array is advanced into the cochlea or into some other cavity went to the body. Of course, this utilitarian value can exist even in embodiments where the actuators are located more proximal the most distal electrode. Any spacing of actuators that can have utilitarian value can be implemented in at least some exemplary embodiments providing the art enable such.

D7, which is the distance from the tip to the portion of the second most distal cutout furthest from the tip can be less than or equal to or greater than 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.25, 3.5, 3.75, 4, 4.25, 4.5, 4.75, 5, 5.25, 5.5, 5.75, 6, 6.25, 6.5, 6.75, 7.0, 7.25, 7.5, 7.75, 8, 8.25, 8.5, 8.75, 9, 9.25, 9.5, 9.75, or 10 mm, or any value or range of values therebetween in 0.01 mm increments (of course, D7 would not equal D6, and would not be values where there would be overlap the length of any given electrode, etc.—these values are simply values that can exist in some embodiments providing that the art enables such—and note that a length of a cutout can be less than or equal to and/or greater than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6. 1.65, 1.7, or 1.75 mm, or any value or range of values therebetween in 0.01 mm increments).

D8 which is the value from the most distal portion of the third electrode from the end to the most distal portion of the second electrode from the end can be any of the values of D6 (and need not be the same-indeed, D6 in practice will often be larger than D8). And note that the aforementioned value of D8 can be for a spacing of any cutout pair detailed herein. Again, the above values are simply exemplary numbers that can exist in at least some exemplary embodiments for any given permutation.

And it is noted that the cutouts/actuators need not be spaced evenly, as will be understood from the above values.

Some embodiments include an electrode array having, within a distance of 5, 6, 7, 8, 9, 10, 11, 12, 13, 14, 15, 16, 17, 18, 18, 19, 20, 21, 22, 23, 24, 25, 26, 27, 28, 29, 30, 31, 32, 33, 34, 35, 36, 37, 38, 39, 40, 41, 42, 43, 44, or 45 mm, or any value or range of values therebetween in 0.1 mm increments, less than or greater than or equal to 2, 3, 4, 5, 6, 7, 8, 9, 10, 11, 12, 13, 14, 15, 16, 17, 18, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 full cutouts and/or actuators. Moreover, in some embodiments, there is only one actuator and/or cutout. In some embodiments, all actuators and/or cutouts are located on one side of the longitudinal axis (aside from cutouts that may exist for the electrodes), while in other embodiments, the actuators and/or cutouts are located on both sides of the longitudinal axis.

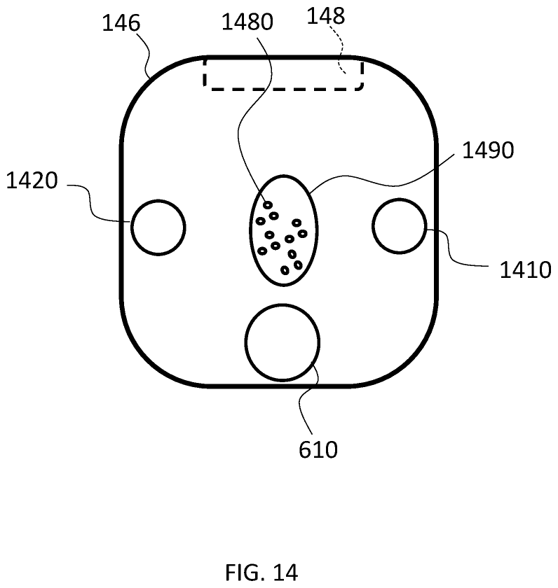

Further, while the embodiments above have been directed towards looking at the electrode array from the side, and the actuators being located at the top or at the bottom, in other embodiments, the actuators can be located in the middle or anywhere between the bottom and the top with respect to the vertical direction. In this regard, FIG. 14 depicts a cross-sectional view of an electrode array taken normal to the longitudinal axis. It can be seen that there is a central cavity 1490 in which electrical leads 1480 are located, concomitant with standard designs for cochlear implant electrode arrays, although it is noted that in some embodiments, the leads and/or the cavity need not be centrally located. Also shown are cross-sections of actuators 1410 and 1420 and 610. Actuator 610 corresponds to the actuators detailed above, although in this embodiment, the actuator is fully embedded in the silicone body of the carrier of the electrode array as shown. In this regard, in this embodiment, the silicone of the carrier completely envelops the actuator 610. Accordingly, instead of utilizing cutouts, at least cutouts that are exposed to the ambient environment, embodiments can include cavities within the silicone of the carrier that contain the actuators and/or other supporting components (e.g., the end plates). In this regard, this is the arrangement shown with respect to actuators 1420 and 1410. The actuators are completely subsumed inside the carrier member. But note that in alternative embodiments, cutouts can also be utilized in a manner concomitant with the arrangements above for the actuators 610 utilizing cutouts. And note that with respect to the embodiments associated with hydrogel, in some embodiments, the hydrogel can actually be integrated into the array. And consistent with the teachings above with regard to the actuators that are inside the carrier body, some embodiments include molding the carrier body around the actuators.

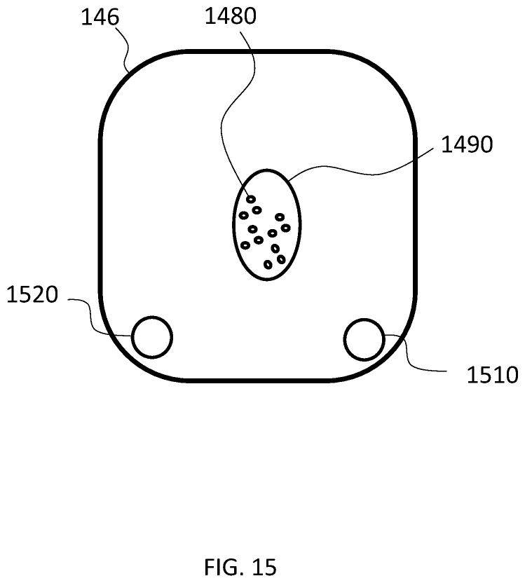

The embodiment of FIG. 14 can have utilitarian value with respect to enabling both horizontal and vertical “curling.” And note that in some embodiments, there might only be one of the two actuators 1420 and 1410. In this regard, one actuator could allow both left horizontal and right horizontal curling, such as in the case of an electrode array that is molded to be biased in one direction and/or in the case of an array assembly where the actuator can expand and/or contract to curl the array in one direction or the other depending on how much expansion and/or contraction there exists. And also positioning the actuators offset can accomplish curling in either direction in some embodiments. By way of example only and not by way of limitation, FIG. 15 shows actuators 1520 and 1510 located at the bottom on either side of the cross-section of the electrode array. Here, for example, if it was desired to curl the array to the left, actuator 1520 could be contracted and/or actuator 1510 could be expanded and vice versa if it was desired to curl the array in the opposite direction. If it was desired to curl the array upward, actuator 1520 and/or actuator 1510 could be expanded and if it was desired to curl the array downward, one or both of those actuators could be contracted. By actuating both of the actuators by the same amount, any twisting about the longitudinal axis that would result from actuating one actuator only would be counteracted, at least with respect to curling in the vertical direction.

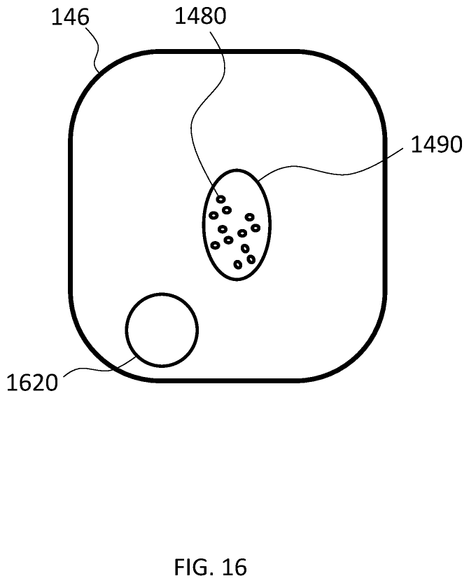

It is noted that the embodiment of FIG. 15 could result in some twisting of the actuator about the longitudinal axis if the array was curled to the left or to the right. This can be owing to the offset nature of the actuators relative to the vertical direction. In some embodiments, this twisting is de minimus or otherwise acceptable, if only because the twisting is temporary owing to the fact that the curling is used simply to snake the array through an area, and the actuator forces that created the curling will ultimately be relieved upon final placement of the array. And corollary to this is that potentially only one actuator could be present, such as that shown in FIG. 16, with actuator 1620, and any twisting that results from the curling of the actuator upwards or downwards could also be de minimis or otherwise acceptable for the reasons detailed above.

Embodiments can enable the actuator to be moved around after implantation. In some scenarios of use, days or weeks or months after implantation, the electrode array may migrate. Alternatively, nerve cells may deteriorate resulting in a less than utilitarian placement of the electrode array at a later date relative to that which was the case in the prior date. By actuating the actuators, which can happen days or weeks or months or years after implantation, the electrode array can be moved within the cochlea. In some embodiments, this can be done without having to access the cochlea or otherwise perform an even minor invasive surgical procedure. In an exemplary embodiment, the implantable component can be controlled to actuate the actuators. This can be done by sending a transcutaneous signal to the implantable component from an external inductance coil by way of example. Corollary to this is that the environment of the electrode array could be changed, such as via the introduction of some form of substance into the cochlea, or by increasing the saline level of the recipient's blood in a controlled manner, etc., or potentially by increasing temperature, etc., so as to re-actuate the actuators. In some embodiments, the actuators can be susceptible to ultraviolet radiation or infrared radiation to control the actuators to move. Magnetic fields could potentially be utilized to actuate the actuators.

In some embodiments, the actuators can be actuated and/or deactuated in a manner so that the electrode array could “worm” its way further into the cochlea or further out of the cochlea. A less exotic use could be to transfer the electrode array from a modiolar wall hugging array to a lateral wall hugging array at some date after implanted. In view of the above, in at least some exemplary embodiments, there is a method that includes actuating and/or the actuating the actuators at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 days or weeks or months or 40 or 50 or 60 or 70 or 80 or 90 or 100, or 125, or 150 or 175 or 200 months or more after implantation where the actuators have not been activated and/or deactivated during that intervening period of time or at least a majority of the time or at least two thirds of the time or at least three quarters of the time. This actuation and or the actuation is executed to purposely move or reposition the electrode array.

By placing the actuator at the offset location, a single actuator could provide curling in both the horizontal and vertical directions. This can have utilitarian value with respect to the fact that the cochlea is a spiral in three dimensions. That is, any array inserted into the cochlea will curve, but will also extend upward (or downward) because the ducts of the cochlea do not spiral in a plane, but spiral upward (or downward) with distance from the basal portion of the ducts. Accordingly, the actuators could be positioned offset from both the horizontal and vertical, but not necessarily with equal offset (there could be equal offset), to achieve curling out of plane that matches the cochlea out of plane features.

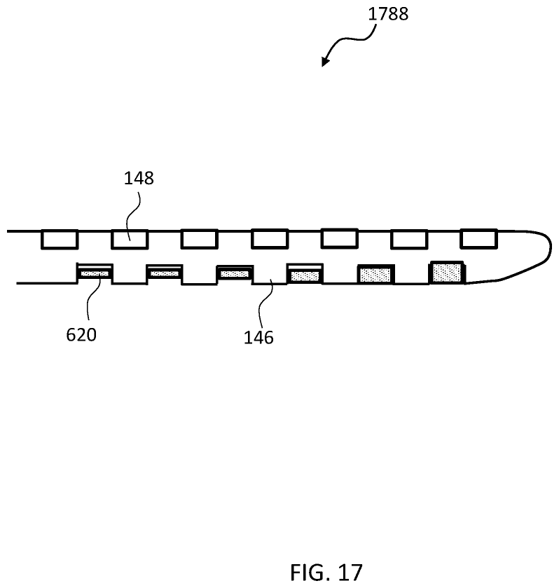

A visual inspection of the cross-sections of FIGS. 14 and 15 and 16 will reveal that the actuators are of different sizes, at least with respect to the cross-sectional diameter shown. This is because the actuators can be smaller or bigger depending on the number of actuators that work in a given plane. This can also be because there might be more curling desired in one plane than the other plane. Accordingly, embodiments include actuators of different sizes depending on the amount of curl, or, more accurately, the amount of force that is desired when the actuators are actuated. And now with respect to FIG. 17, there can be an electrode array portion 1788 that has actuators 620 of different diameters/cross-sectional areas (in the plane normal to the longitudinal axis of the array) so as to achieve different local radius of curvatures. In this exemplary embodiment, owing to the greater diameter of the actuators, all other things being equal, a greater force output will result with respect to the greater diameters the actuators relative to those more proximal. In this exemplary embodiment, at least when the actuators are actuated by their fullest amount (whether that is to obtain the curl or to eliminate the curl, where upon the relaxation of the actuators, the array curls), the local radius of curvature increases with location along the array towards the distal end (if only in a digital manner) owing to the progressively larger actuators. Thus, a more “tighter” curl can be achieved at the distal end owing to the larger actuators, thus more closely aligning with the higher radius of curvatures of the more difficult portions of the cochlea.

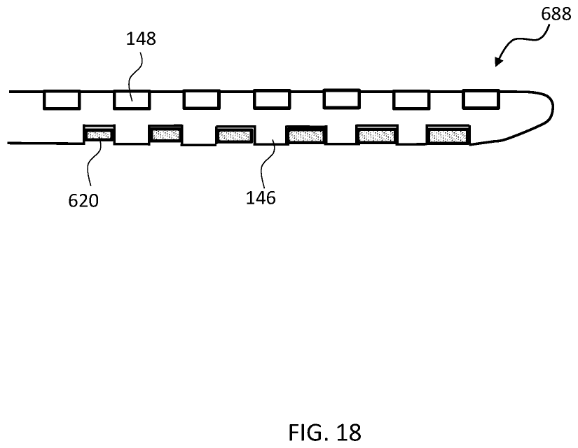

FIG. 18 presents another exemplary embodiment that utilizes different sized actuators to obtain different features associated with the curling of the array. Here, instead of the actuators having different cross-sectional areas, the actuators have different lengths. The longer the length, the greater the local radius of curvature proximate that actuator. And with regard to FIG. 17 and FIG. 18, in an exemplary embodiment, the actuators can be utilized to achieve the curling of FIG. 10, which has a radius of curvature that increases with distal location.

And note that the features of FIGS. 17 and 18 can be combined. There can be actuators of varying length and varying diameters that vary the local force and thus the local radius of curvatures. And note that a given actuator can have a different length and/or a different diameter than that of another actuator. Also, the way that the actuators are staggered can also be varied to achieve the different local radius of curvature. In this regard, while in some embodiments, the actuators can be arrayed in a standard and even distribution that is repetitive, where the distances from each actuator are not the same and/or the distances from the center of area and/or the center of mass of the same, in other embodiments, the actuators can be arrayed in a nonstandard and not even distribution that is not repetitive, where the distances from each actuator are not the same and/or the distances from the center of area and/or the center of mass are not the same.

Also, distances of the actuators from the longitudinal axis can be different. In an embodiment, any one or more actuator can have a center of mass in a relaxed state that is less than, equal to and/or greater than 0.05, 0.075, 0.1, 0.125, 0.15, 0.175, 0.2, 0.225, 0.25, 0.275, 0.3, 0.325, 0.35, 0.375, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65 or 1.7 mm or any value or range of values therebetween in 0.001 mm increments, and these distances can be different for one or more or all of the actuators (any actuator can have any of these distances).

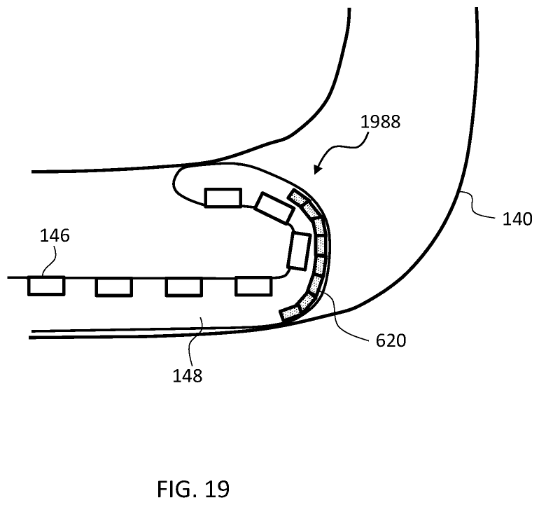

Some exemplary embodiments can utilize the actuators to reverse the occurrence of tip fold over. In at least some scenarios of insertion of cochlear implant electrode arrays into the cochlea, the tip can experience a fold-over condition, such as that shown in FIG. 19. This “curvature” of the electrode array is not desirable, and does not represent a curled array that would exist in a proper implantation scenario. Instead, this depicts an array that is essentially wedged inside the duct of the cochlea, and further insertion into the duct of the cochlea will further exasperate this tip fold-over owing to the continued narrowing of the duct from the basil location to the apical location. Typically, the only way to alleviate fold-over, if possible, is to withdraw the electrode array from the cochlea at least partially, and then attempt to re-insert the electrode array in the cochlea by pushing electrode array further into the cochlea. This sometimes does not work, and in fact in many instances the surgeon may not necessarily be aware the tip fold-over has occurred. Accordingly, in at least some exemplary scenarios of implantation with respect to the prior art, the recipient of the cochlear implant electrode array go through life with a folded over electrode array.

Conversely, embodiments that utilize the actuators 620 can potentially alleviate this tip fold-over condition. In the embodiment of FIG. 19, can be seen there is a plurality of actuators 620 connected to each other that are arranged so that when the actuators are actuated, the array, at least approximate those components, will straighten out. The idea being that the actuation of the actuators and the accompanying straightening of the election array will results in the tip of the array being pulled forward, and thus straightened. This could for example be performed if the tip fold over condition is discovered after implantation, through, for example, identifying anomalies in the function or performance of the device, and methods include doing so and then using the actuators to remedy/eliminate or at least reduce the amount of fold over.

In the embodiment shown in FIG. 19, the actuators are arrayed end-to-end and in contact with each other, and in this regard, there is a single cutout that extends across multiple electrodes as can be seen. That said, in some alternative embodiments, separate cutouts for separate actuators can be utilized. And note that in some embodiments, it may only be that there is one actuator and/or two or three actuators that are utilized to alleviate tip fold-over. The arrangement geometry and the numbers of actuators that will be utilized can be any that can enable the teachings detailed herein providing that the art enable such.

In view of the above, it can be seen that in an exemplary embodiment, there is an electrode array, such as a cochlear implant electrode array, that comprises a plurality of electrodes, and an electrode carrier carrying the plurality of lectures. This carrier can be made out of silicone or any other biocompatible material that can enable the teachings detailed herein. In at least some exemplary embodiments, the electrode carrier includes an artificial muscle component. The artificial muscle can be represented by element 620 detailed above. In an exemplary embodiment, the electrode arrays configured so that the artificial muscle component expands upon a trigger event, where, consistent with the concept of a muscle, the expansion is a result of relieving of the stimulus that caused the contraction of the muscle. That is, in an exemplary embodiment, the trigger event is the relieving of the stimulus that cause the contraction of the muscle. In other exemplary embodiments, the artificial muscle component is configured to contract upon a trigger event. Consistent with the teachings above, in at least some exemplary embodiments, the artificial muscle component is a polymer with a preferred direction of a polymer chain thereof, the preferred direction being adopted upon a trigger event (and in some embodiments, upon the relief of the trigger event, the preferred direction may be counteracted upon the relief of the trigger event, which could be because of the way that the electrode array is molded, where, for example, if the polymer cannot maintain the preferred direction in the absence of the stimulus, memory of the electrode array can counteract the preferred direction. And consistent with at least some exemplary embodiments above, the artificial muscle component is a hydrogel-based component.

As seen above, in at least some exemplary embodiments, the electrode array includes a plurality of artificial muscle components distributed at intervals (even or non-even) along a longitudinal direction of the electrode array.

In some embodiments, the electrode array is configured so that a local radius of curvature of the electrode array can be controlled to have at least three different values each separated from each other by at least 5% of the minimum controlled local radius of curvature. Accordingly, by way of example, the artificial muscle could be configured to contract to its most contracted state, to yield a local radius of curvature of r1. Then, the artificial muscle could be controllably relaxed from its most contracted state to a state where the muscle is only 75% contracted, for example, to yield a local radius of curvature of r2, where r2 is at least 1.05 times r1 or no more than 0.95 times r1. Then, the artificial muscle could be controllably relaxed from its most contracted state to a state where the muscle is only 50% contracted, for example, to yield a local radius of curvature of r3, which would be at least 1.10 times r1 or no more than 0.9 times r1 and so on.

In some embodiments, the electrode array is configured so that a local radius of curvature of the electrode array can be controlled to have at least 2, 3, 4, 5, 6, 7, 8, 9, or 10, or any value or range of therebetween in 1 increment different values each separated from each other by at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50%, or any value or range of therebetween in 1% increments of the minimum controlled local radius of curvature.

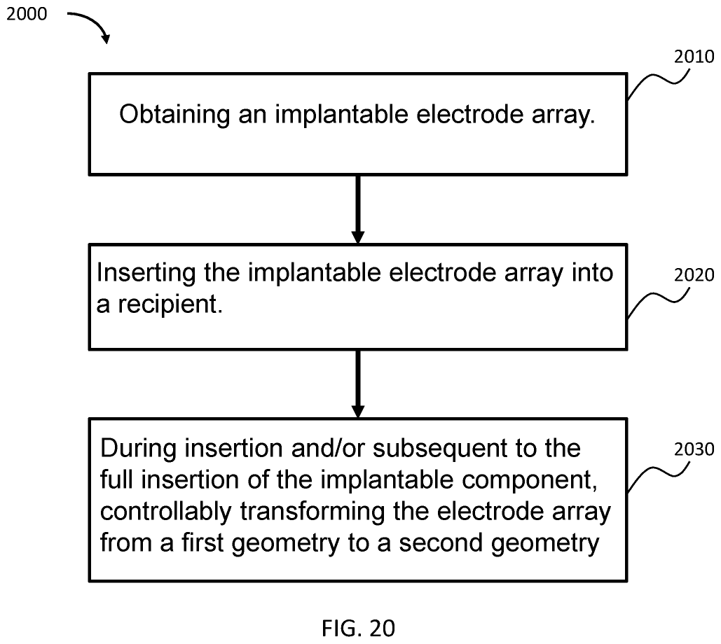

Embodiments include methods. FIG. 20 presents an exemplary flowchart for an exemplary method, method 2000, that includes method action 2010, which includes the action of obtaining an electrode array that is implantable in the human. By way of example, this can be executed by obtaining implant 144 in its entirety, or at least the elongate array 118 thereof, or at least the electrode array 190 (with reference to FIG. 1B). Method 2000 further includes method action 2020, which includes inserting the implantable component into a recipient (which can be achieved by implanting the electrode array 190 into the cochlea (i.e., implanting the intra-cochlea region 188). Method 2000 also includes method action 2030, which includes during insertion and/or subsequent to the full insertion of the implantable component, controllably transforming the electrode array from a first geometry to a second geometry without any of external force relief, external pressure relief and reaction force controlling transformation.

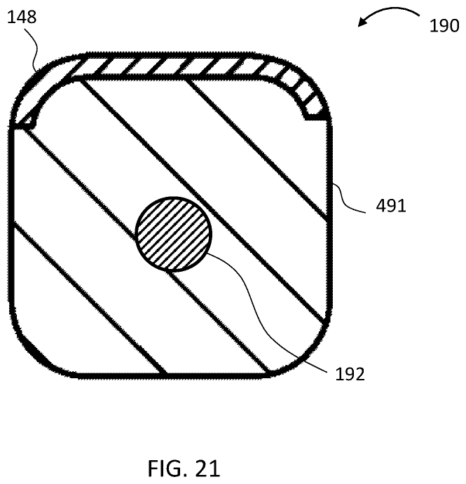

By without external force relief, it is meant that the transformation does not start with and/or is not established from the removal or relaxation of a force applied to the electrode carrier, such as, for example, that which results when a so-called stylet is removed from the intra-cochlear portion of the electrode array. In this regard, FIG. 21 depicts an exemplary embodiment of an electrode array that includes a stylet 192. Method 2000 can be practiced with or without electrode arrays that utilize a stylet as long as the aforementioned feature of the transformation results in addition to any transformation that results from the removal of the stylet (removal in part or removal in full).

By without external pressure relief, it is meant that the transformation does not start with and/or is not established from the removal or relaxation of a pressure applied to the electrode carrier, such as, for example, that which results when the electrode array is removed from a so-called insertion sheath. By without mass transfer, it is meant that the transformation does not start with and/or is not established from a component of the electrode array being transferred therefrom, such as, for example, that which results from a portion of the electrode array dissolving.

By without reaction force, it is meant that the transformation does not start with and/or is not established from a force that is reactive against a surface (e.g., a surface of the recipient, a surface of the electrode array, etc.), such as, for example, that which results when a portion of the electrode array springs out or otherwise extends to a location in contact with a portion of the cochlea so as to “push” the electrode array from a position that existed prior to the reaction force.

By without net energy transfer, it is meant that the transformation does not start with and, in some instances, does not result from, a net change in energy transfer to or from the electrode array, such as, for example, that which results when a portion of the electrode array heats or cools from a temperature thereof at the time that the electrode array was fully inserted into the cochlea.

In an exemplary embodiment, the action of transforming from the first geometry to the second geometry is executed without moving any component relative to the implant that initiates and/or establishes transformation. In an exemplary embodiment, no stylet is moved or otherwise withdrawn such that the transformation is initiated. In an exemplary embodiment, no insertion sheath (or any sheath for that matter) is moved or otherwise withdrawn such that the transformation is initiated.

It is noted that the first geometry is not necessarily a geometry corresponding to the initial insertion geometry. The point is that the first geometry need not necessarily be the insertion geometry. In fact, in scenarios where the electrode array is inserted such that the outer side of the curved array (the side facing away from the “center” about which the curved array extends/the side facing away from the electrodes in the case where the electrodes do not completely extend about the outer circumference of the electrode array) and/or the tip of the array contacts the lateral wall of the cochlea, the force of insertion will quite frequently drive the electrode array into a geometry away from the insertion geometry (the driven geometry being the aforementioned first geometry in at least some exemplary embodiments). Conversely, in embodiments where the insertion process is such that the curved array extends into the cochlea during the insertion process such that the curved array does not come into contact with any of the walls in the cochlea, the insertion geometry can be the aforementioned first geometry.

In an exemplary embodiment, the electrode array is configured such that the time to change from the third geometry to the first and/or second geometry is sufficient for the surgeon to insert the electrode array into the cochlea in a geometry corresponding to and/or substantially corresponding to the third geometry and/or in a geometry “between” the third geometry and the first geometry. In an exemplary embodiment, this provides sufficient time for the tip of the electrode array to reach the back of the basil turn of the cochlea, and thus avoid tip fold-over.

In an exemplary embodiment, the action of transforming from the first geometry to the second geometry is entirely a result of one or more artificial muscles that is/are an integral part of the electrode array. This as opposed to an external device or a part of the implant separate from the array. In some embodiments, the action of transforming from the first geometry to the second geometry is executed without moving any component relative to the implant that does not become implanted with the array that initiates or results in the transformation.

Consistent with the teachings above, in an embodiment, the electrode array is a cochlear electrode array, the action of inserting the implantable component into the recipient includes inserting the electrode array into a cochlea, the first geometry is a curved geometry resulting at least in part from the curvature of the cochlea (e.g., the electrode array contacts the lateral wall of the cochlea, thus deforming the electrode array from the geometry that was the case prior to the electrode array contacting the lateral wall of the cochlea), and the second geometry is a curved geometry having an average radius of curvature that is lower than that of the first geometry (e.g., the average radius of curvature over the first distance noted above, or the average radius of curvature over the distance extending from the inside wall of the cochlea at the point where the electrode array enters the cochlea to the tip of the cochlea, etc.). In an exemplary embodiment, the average radius of curvature differs by less than, equal to or more than 5, 10, 15, 20, 25, 30, 35, 40, 45, or 50%, or any value or range of values therebetween in 1% increments, from that of the first geometry (using the first geometry as the denominator).

In view of the above, in an exemplary embodiment of method 2000, where the implantable component is a cochlear electrode array, the action of inserting the implantable component into the recipient (the action of inserting the electrode array into the cochlea) entails inserting the implantable component into a cochlea of a recipient in a third geometry, where the first geometry is a curved geometry resulting at least in part from the curvature of the cochlea (e.g., due to resistance from the lateral wall from the insertion geometry), and the third geometry is one of a substantially straight geometry or a negatively curved geometry relative to the curved geometry of the first geometry (it is noted that this third geometry is not necessarily the insertion geometry-more on this below). In this regard, in an exemplary embodiment, method 2000 entails obtaining an electrode array, which, in at least some exemplary embodiments, has a relaxed curved state such that, with respect to the frame of reference of FIGS. 1B and 2, the electrode array curves in a counter-clockwise direction. Put another way, the electrode array curves such that the electrodes “see” more of each other. Method 2000 further entails deforming the electrode array from this relaxed curved state to a straight/substantially straight configuration (or the method entails obtaining the electrode array in this straight/substantially straight geometry). This can be done by actuating or deactivating the actuators. In an alternate embodiment, method 2000 further entails deforming the electrode array from this relaxed curved state to a negatively curved state (or obtaining the electrode array in this negatively curved state), where the electrode array curves in a clockwise direction with respect to the frame of reference of FIGS. 1B and 2. Put another way, the electrodes see “less” of each other, just as the curvature of the Earth causes one to see less of structures because the curvature of the Earth eclipses some or all of the structures. In an exemplary embodiment, there can be utilitarian value with respect to deforming the electrode array to this negative curvature because, in at least some exemplary embodiments, the time between the release of the forces applied to the electrode array to place the electrode array and/or to hold the electrode array in this negatively curved geometry and the time at which the electrode array returns to its relaxed state is longer (e.g., due to, for example, the increased degree of strain of the viscoelastic material associated with deforming it further from its relaxed configuration) than that which would be the case with respect to the time between the release of forces applied to the electrode array to place the electrode array and/or to hold the electrode array in a substantially straight geometry and the time in which the electrode array returns to its relaxed state. In an exemplary embodiment, the former is at least about 1.1, 1.2, 1.3, 1.4, 1.5, 1.6, 1.7, 1.8, 1.9, or 2 times or more than the latter.

The negative curvature can be a result of contracting the artificial muscles by the maximum amount, where contracting the muscles to less than the maximum amount yields a straight configuration, and so on.

Thus, in an embodiment, the method 2000 is such that the electrode array is a cochlear implant electrode array, the action of inserting the implantable component into the recipient includes inserting the electrode array into a cochlea of a recipient in a third geometry, the first geometry is a curved geometry resulting at least in part from the curvature of the cochlea, and the third geometry is one of a substantially straight geometry or a negatively curved geometry relative to the curved geometry of the first geometry.

Further, in an embodiment where the array is a cochlear implant electrode array, the action of inserting the implantable component into the recipient includes inserting the electrode array into a cochlea of a recipient in the first geometry, the transformation from the first geometry to the second geometry takes less than 45, 40, 35, 30, 25, 20, 15, 10, or 5 seconds, or any value or range of values therebetween in 1 second increments.

In an embodiment where the cochlear implant electrode array is a curved cochlear electrode array, the action of inserting the array into the recipient includes inserting the electrode array into a cochlea of a recipient in a third geometry, the second geometry is a curved geometry, the third geometry is one of a substantially straight geometry or a negatively curved geometry relative to the curved geometry of the second geometry and the second geometry is closer to a relaxed state of the electrode array than the third geometry.

Embodiments can include an implantable apparatus, comprising an electrode array including a plurality of electrodes and a carrier carrying electrodes. With respect to this embodiment under discussion, wherein the electrode array is configured so that, during and/or after insertion into a recipient, a local radius of curvatures at a first location and a second location more distal than the first location can be simultaneously controllably changed relative to one another. By “controllably changed” it is meant that the local radius of curvature can be achieved in an intentional and at least semi-precise manner. This arrangement where the different local radius of curvatures can be simultaneously controllably changed differentiates from a stylet, where removal of the stylet changes the radius of curvatures in a serial matter as the stylet is withdrawn. This arrangement also distinguishes from shape changing materials such as a viscoelastic material or a material that changes shape owing to temperature change in that those are not control. Instead, those result from any evolution from the first state to a second state without any true control, at least with respect to utilizing the electrode array to control the change.

Consistent with the teachings above, in an exemplary embodiment, the local radius of curvature at the first location is controlled by one or more actuators located, relative to a longitudinal direction of the electrode array, between two electrodes. In an exemplary embodiment, the local radius of curvature at the first location is controlled by relieving a tension force in the carrier, the relieving of tension increasing the local radius and/or establishing the local radius of curvature, respectively. In an embodiment, the local radius of curvature at the first location is controlled by applying an expansion force in the carrier, the applying of the expansion force increasing the local radius and/or establishing the local radius of curvature, respectively.

It is noted that any of the features associated with the first location can be associated with the second location and vice versa.

Continuing further with this exemplary embodiment, in an exemplary embodiment of this exemplary embodiment, the local radius of curvature at the first location is controlled by actuators in the carrier, wherein upon turning off the actuators, the local radius of curvature is increased and/or the local radius of curvature is established, respectively. By turning off, it is meant that the actuators have a natural at rest state. With respect to the above noted polymers, the “off” state can be the state where the preferred direction of the polymer exists in the environment of the cochlea, and no additional stimulus is needed to maintain that state. Conversely, the stimulus that is utilized to turn “on” the actuators would be temporary. The relief of that stimulus would result in the transformation of the electrode array to obtain a radius of curvature at a locality as detailed herein. In some embodiments, the local radius of curvature at the first location is controlled by actuators in the carrier, wherein upon turning on the actuators, the local radius of curvature is increased and/or the local radius of curvature is established, respectively.

In some embodiments, the electrode array is configured so that, after insertion into a recipient, beyond that which results from tissue of the recipient causing such change and/or adoption, (i) a plurality of local radius of curvatures at a plurality of spatially separate locations, including the first location and the second location, can be controllably changed to be different from one another and/or (ii) the plurality of local radius of curvatures can be controllably obtained at the plurality of spatially separate locations from no radius of curvature at the respective plurality of spatially separate locations.

In some embodiments, the plurality of spatially separate locations include at least 2, 3, 4, 5, 6, 7, 8, 9, 10, 11, 12, 13, 14, 15, 16, 17, 18, 19, or 20 or more, or any value or range of values therebetween in 1 increments.

Embodiments can be such that with respect to a closest distance of the most distant electrodes of the electrode array, the first location is within 0.1, 0.15, 0.2, 0.25, 0.3, 0.35, 0.4, 0.45, 0.5, 0.55, 0.6, 0.65, 0.7, 0.75 mm, or any value or range of values therebetween in 0.01 increments of that distance from a distal tip of the array, and thus the second, and if present, the third, fourth, fifth, sixth locations, etc., would also be within that distance.

Again, FIG. 6 can represent how an array that is moulded in the curved configuration and can be “pulled” straight by contracting the artificial muscles, which can then facilitate insertion into the cochlea. To recap, after insertion and/or during insertion (e.g., as the electrode array reaches the first turn of the cochlea), the artificial muscles can be relaxed, and thus permitted to lengthen, to allow the electrode array to return to its curved shape. And in an embodiment, the artificial muscle(s) proximate the tip/more distal than others, could relax first, and then progressively more proximal artificial muscles can relax in a serial manner (for example only) as the array progresses into the cochlea.

Embodiments can be implemented where actuators are present, which can be turned off as the electrode array is inserted in the cochlea to allow the array to adopt its perimodiolar shape. Actuators can be placed at any location to enable pulling of the array straight upon actuation. At locations with greater curvature there can be utilitarian value to have actuation (greater change in length of the actuator). More on this below. And in some embodiments, the converse is also implemented (instead and/or in addition to the above), where the array is moulded straight and the actuators induce curvature as the electrode is inserted in the cochlea. In this approach the actuators could be placed selectively (not along the whole length). The disadvantage of the moulded straight approach is that the actuators have to be actuated permanently once implanted. This is achieved in some embodiments when the cochlea environment caused the actuation to occur, such as, for example, warming to 37C and/or interfacing with the body fluid.

In both embodiments, different levels of actuation at different locations along the length of the array can be achieved by either different actuators at different locations (different materials; different levels of actuation), or a common actuator which can generate different levels of actuation e.g., due to different applied current. So, for instance, the basal actuators may be at 50% actuation; the mid electrode one at 75%; and the tip actuators at 100%. Another way of achieving this would be to use a hydromorphic material on the outer surface which expands in perilymph to create the curved shape. Thus, embodiments can utilize different levels of actuation to obtain different geometries, more accurately, different local geometries, of the electrode array. (Of course this can be done to achieve different global geometries of the electrode array.) The example of 50%, 75%, and 100% actuation has been described above. In some embodiments, additional degrees and/or graduations of actuation can be achieved. For example, a given actuator could have two or three or four or five or six or seven or eight or nine or 10 or more graduations of actuation, all having a different extension and/or contraction and/or force output (push or pull). In at least some exemplary embodiments, these different levels are based on the ability to control triggers or otherwise to control the environment, or otherwise to control stimulus applied to the electrodes. By way of example, electricity can be utilized to control the actuators. Different levels of voltage can be utilized to achieve these different levels of actuation. Moreover, some embodiments include utilizing actuators that are controlled in an analog manner. By way of example, the actuators can be actuated at what would be infinite graduations save for the fact that such is impossible and is otherwise limited by the finite nature of any control regime or otherwise physics or engineering.

In some embodiments, such as where the mechanism of shape forming is contained within the body of the electrode array (within the carrier for example) and/or between electrode pads, this can allow tuning of electrode shape by activating actuators to create more or less curvature as required. In some embodiments, this is used to tune the electrode array to better match the curvature of cochleas of different sizes-given the known variation person to person. Accordingly, in an exemplary embodiment, there includes a method where the size and/or shape of a cochlea of a specific human being is determined, and/or a size and/or shape of a cochlea of a specific human being is estimated based on statistical data. An electrode array is then fabricated based on that data, or more specifically, actuators are selected and/or positioned to achieve an ultimate shape of the electrode array after insertion that is customized to that particular cochlea. The size and/or strength actuators can be selected, and/or the number of actuators and/or the placement of the actuators can be selected to achieve the given curvature. Accordingly, embodiments include making an electrode array having any one or more of the features detailed herein based on data associated with a specific person. Moreover, data is associated with a group of people that have similar characteristics can be compiled and based on that data, utilizing statistical analysis, different designs for different electrode arrays utilizing the various actuator arrangements detailed herein can be developed. That is, a specific person need not necessarily be identified for the design. Instead, a class of people can be identified, and then the electrode array can be designed to meet that class. Later, a physician or the like can identify which class of people a given potential recipient would be a part, such as, for example, using an X-ray system and/or a CT scan or some other non-invasive scanning system, or using statistical data based on other physical features, and then, based on that identification of the classification, select a specific design of the electrode array that has been tuned according to the teachings detailed herein to be implanted into that person.

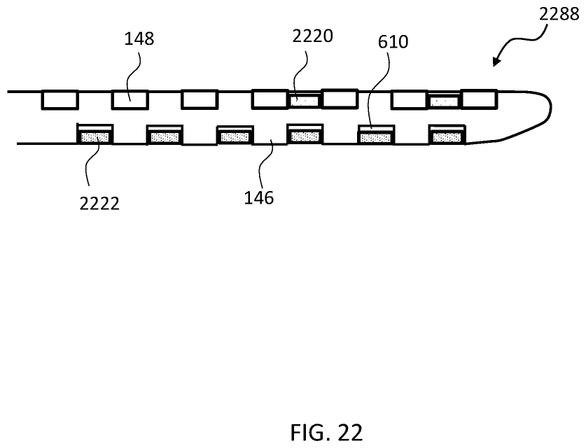

Embodiments above have focused on the concept of utilizing push actuators or pull actuators in a given electrode array. Embodiments can also include the combination of push and pull actuators in a single array. In this regard, FIG. 22 depicts an exemplary cochlear implant electrode array 2288 that includes push actuators 2220 and pull actuators 2222. These designations refer to the state to the actuator when the actuators are turned on. Thus, the configuration shown in FIG. 22 depicts those actuators on one. As the carrier of the electrode array is a molded curved actuator. The activation of the actuators respectively pushes and pulls the upper portion and the bottom portion to result in an electrode array that is in a straight configuration as shown in figure. The activation of the actuators results in the electrode array curling.

And note that in the embodiment shown in FIG. 22, there are only two push actuators 2220, and these are located between the electrodes shown. In this exemplary embodiment, there is utilitarian value with respect to having the extra actuators at the tip so as to achieve a greater curl at the tip/to achieve a greater local radius of curvature at the tip then that which results more proximal from only the pull actuators 2222. This is an example of how actuators can be arrayed differently to achieve different geometric configurations. And note that in other embodiments, the push actuators can be between each electrode or could be between more than just the electrode pairs shown. The converse can also be the case. There could be fewer pull actuators 2222 than the spaces between the electrodes. To be clear, any number of actuators could be used providing that the art enables such. As few as one can be used, if only one part of the array is desired to have the controllable curvature.

In an embodiment, the actuators are located only in the distal portions of the array. In an embodiment, the actuators are located over a distance that is less than 80, 75, 70, 65, 60, 55, 50, 45, 50, 35, 30, 25, 20, 19, 18, 17, 16, 15, 14, 13, 12, 11, 10, 9, 8, 7, 6 or 5% or less or any value or range of value therebetween in 1% increments of the distance from the two furthest electrodes as measured from the most distant portions of the electrodes. In an embodiment, the aforementioned distances of the actuators starts at the distal end (the distances of the actuators is measured from the most distant portions thereof). The measurement can be taken when the array is straight or curved, and is relative to the longitudinal axis.

Exemplary actuators can be found in Park and Kim's publication, entitled Hydrogel-Based Artificial Muscles: Overview and Recent Progress, published in Advanced Intelligence Systems, in 2020.

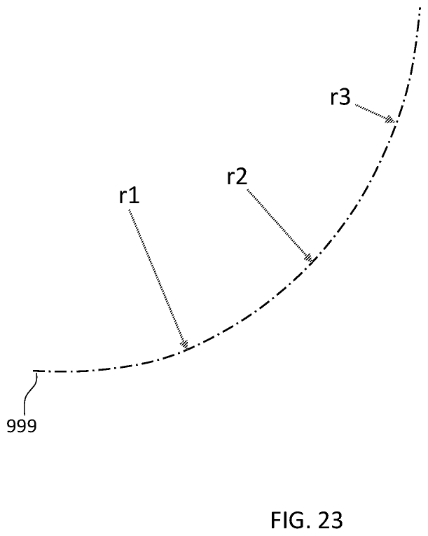

As noted above, embodiments can provide an array with controllable different local radius of curvatures. FIG. 23 duplicates the curve of FIG. 10A that is associated with the longitudinal axis of the array in the configuration shown in FIG. 10. FIG. 23 shows three enumerated radius of curvatures. Radius of curvature r1 is larger than radius of curvature r2, and radius of curvature r2 is larger than radius of curvature r3. Here, the radius of curvature r3 would be the minimum radius of curvature. Thus, r2 would be at least 1.05 times r1, and r3 would be at least 1.10 times r1 (or r2 would be at least 1.10 times r1, and r3 would be at least 1.05 times r1). And in an exemplary embodiment, the electrode array can be controlled to have these values when the electrode array is in free space (this can be done for any of the configurations herein). That is, the electrode array is in a fluid medium and is not constrained by a solid object. This can be a convenient way to test the electrode array as to whether or not it meets these values. In at least some exemplary embodiments, these values are taken on a plane for at least within two parallel planes bounded by each other by no more than 0.5, 1, 1.5, 2, 2.5, 3, 3.5 or 4 mm.

In an exemplary embodiment, there can be two, three, four, five, six, seven, eight, nine, 10, 11, 12, 13, 14, 15, 16, 17, 18, 19 or 20 or more or any value or range of values therebetween in 1 increment locations along the electrode array that have local radii of curvature that can be controlled utilizing the teachings detailed herein.

In an exemplary embodiment, at least some of the actuators serve a dual purpose as a drug eluting devices. In this regard, some of the substances that are used to form the actuators can be “impregnated” or otherwise charged with a therapeutic substance, such as a drug, or a steroid, etc. Thus, after implantation, over time, the therapeutic substance will elute from the actuator material into perilymph in the cochlea, by way of example.

Embodiments can utilize feedback routines to determine an amount of curvature or otherwise “sense” a position of the electrode array. In an exemplary embodiment, resistance could be measured and used as a gauge to evaluate resistance or the like to curving, which can give an indication of the position of the electrode array. Alternatively, known quantities can be utilized to estimate, based on statistics, the geometry and/or the positions of the actuators. Temperature sensors can be inserted into the cochlea otherwise combined with the electrode array, where, owing to the fact that in some embodiments, temperature can trigger otherwise control the actuation of the actuators. Knowing the temperature can give a satisfactory indication of the level of actuation of the actuator. Further, in some embodiments, resistive heaters can be included in the electrode array or otherwise positioned proximate the electrode array, which can be used to heat the local environment and/or the material of the actuators to cause actuation. Embodiments can utilize impedance values on the electrodes to determine position and/or orientation or geometry of the electrode array after it is located in the cochlea.

Indeed, in an exemplary embodiment, impedance between electrodes can be measured to potentially estimate local radius of curvatures between two or more given electrodes. To determine any one or more of these values providing that the art enable such. EcoG and/or impedance spectroscopy can be used to determine any one or more of these features. Upon determining such, the electrode array can be repositioned by reactivating and/or deactuating the actuators, or, alternatively, can be determined to be at a utilitarian position, whereupon the actuators will not be reactivated or deactivated.

This feedback/these measurements can be used as a basis to make additional adjustments or to halt further adjustment.

Note also that in at least some exemplary embodiments, impedance in the electrical leads or otherwise the circuit that powers the actuators, in the case of electrically driven actuators, can be utilized to estimate or otherwise determine position and/or geometry of the electrode array. This could be incorporated into a feedback regime that can provide indications as to position and/or geometry of the actuator.

In an exemplary embodiment, no CT scans and/or x-rays and/or MRI scans are utilized within 1, 2, 3, 4, 5, 6, 7, 8, 9 or 10 hours or days or weeks of the cochlear implant electrode array implantation. In an exemplary embodiment, feedback is utilized to determine positional features of the electrode array and/or geometry the electrode array in real time without external imaging devices.

In some embodiments, electrical leads extend to the given actuators to power the actuators. In other embodiments, activation of the electrodes at values that are higher than that which would otherwise be seen during normal operation can be utilized to actuate or de-actuate the actuators. In this regard, a current that would produce an uncomfortable, including a very uncomfortable, reaction in the recipient were the recipient conscious, can be utilized to actuate and/or deactuate the actuator. Because level of current would never be used during normal use, there is no danger of causing the actuators to actuate after implantation, at least not by accident.

And to be clear, the actuators detailed herein are part of the cochlear implant electrode array. They remain implanted in the electrode array for the life of the actuator, or at least until they dissolve or otherwise dissipate in the case of bio-absorbable materials, if such is used. In an exemplary embodiment, a given actuator moves in a one to one relationship, or effectively one-to-one relationship, with the closest electrode of the electrode array. There could be some movement with respect to flexing of the carrier body or the changes in curvature of the electrode array, but for all intents and purposes, the actuators move with the electrodes of the electrode array. This is distinct from insertion tools or otherwise insertion robots or the like that are separate from the electrode array. Accordingly, embodiments include fully implanted electrode arrays that include the actuators and or methods that include implanting the actuators in the recipient and not removing the actuators or assemblies of which the actuators are part after the election array is implanted.

In at least some exemplary embodiments, the actuators cannot come into contact with tissue of the recipient, or any material of the recipient, other than perilymph, at least not until potentially bony growth or fibrous tissue potentially encapsulates the electrode array. In an exemplary embodiment, the actuators may slide along the lateral wall the cochlea and/or the modiolus wall, and/or may contact such, but do not grip or otherwise exert a force against the tissue. Corollary to this is that the actuators may come into contact with the end wall of the cochlea during insertion through the cochleostomy, but again, the actuators do not impart force onto any tissue. In an exemplary embodiment, all force or at least substantially all force generated by the actuators is imparted in the longitudinal direction to change the shape of the electrode array.

In an exemplary embodiment, there is an exemplary method, comprising obtaining a curved electrode array, accessing an interior of the cochlea, inserting the electrode array into the cochlea in a deformed state deformed from a relaxed, unrestrained, curved state, wherein at least a portion of the electrode array remains substantially in the same deformed state after entry into the cochlea. This exemplary method further includes surgically closing the surgical opening of the recipient after accessing the cochlea. This method is executed such that all components entering the cochlea after the action of accessing the interior of the cochlea remain in the cochlea after the action of surgically closing the surgical opening. In this regard, no stylet enters the cochlea (indeed, no stylet is used). It is noted that a stylet that is located entirely within the electrode array is still located in the cochlea if the portion of the array containing the stylet enters the cochlea. Still further, in this regard, no insertion sheath enters the cochlea. Still further, in this regard, there are no dissolvable materials that would dissolve into the fluid of the cochlea, which in turn would be dissolved into other portions of the body, thus leaving the cochlea.

It is noted that some and/or all of the teachings detailed herein can be used with a hearing prosthesis, such as a cochlear implant. That said, while the embodiments detailed herein have been directed towards cochlear implants, other embodiments can be directed towards application in other types of hearing prostheses, such as by way of example, other types of electrode arrays used in medical devices (e.g., pacemakers, nerve stimulators, deep brain stimulators etc.). Indeed, embodiments can be utilized with any type of medical device that utilizes an implanted electrode array, or even a non-implanted array. Still further, the teachings detailed herein are not limited to electrode arrays, but can be utilized with any implant providing that the teachings detailed herein and/or variations thereof have utilitarian value.

It is noted that any disclosure with respect to one or more embodiments detailed herein can be practiced in combination with any other disclosure with respect to one or more other embodiments detailed herein. Also, embodiments include embodiments where any one or more features herein are explicitly excluded from combination with any one or more features.

It is noted that some embodiments include a method of utilizing the apparatuses and systems that have one or more or all of the teachings detailed herein and/or variations thereof. In this regard, it is noted that any disclosure of a device and/or system herein also corresponds to a disclosure of utilizing the device and/or system detailed herein, at least in a manner to exploit the functionality thereof. Further, it is noted that any disclosure of a method of manufacturing corresponds to a disclosure of a device and/or system resulting from that method of manufacturing. It is also noted that any disclosure of a device and/or system herein corresponds to a disclosure of manufacturing that device and/or system. Moreover, any disclosure of a method action herein also corresponds to a system and/or a device for executing that method action. Also, any disclosure of a device and/or system herein corresponds to a disclosure of a method of using that device and/or system, and a method of manipulating that device and/or system using the features disclosed herein.

While various embodiments have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

1. An electrode array, comprising:
a plurality of electrodes; and
an electrode carrier carrying the plurality of electrodes, wherein
the electrode carrier includes an artificial muscle component.

2. The electrode array of claim 1, wherein:
the electrode array is configured so that the artificial muscle component expands upon a trigger event.

3. The electrode array of claim 1, wherein:
the artificial muscle component is configured to contract upon a trigger event.

4. The electrode array of claim 1, wherein:
the artificial muscle component is a polymer with a preferred direction of a polymer chain thereof, the preferred direction being adopted upon a trigger event.

5. The electrode array of claim 1, wherein:
the artificial muscle component is a hydrogel-based component.

6. The electrode array of claim 1, wherein:
the electrode array includes a plurality of artificial muscle components distributed at intervals along a longitudinal direction of the electrode array.

7. The electrode array of claim 1, wherein:
the electrode array is configured so that a local radius of curvature of the electrode array can be controlled to have at least three different values each separated from each other by at least 5% of the minimum controlled local radius of curvature.

8. A method, comprising:
obtaining an implantable electrode array;
inserting the implantable electrode array into a recipient; and
during insertion and/or subsequent to the full insertion of the implantable component, controllably transforming the electrode array from a first geometry to a second geometry without any of:
external force relief controlling the transformation;
external pressure relief controlling the transformation; and
reaction force controlling the transformation.

9. The method of claim 8, wherein:
the action of transforming from the first geometry to the second geometry is entirely a result of one or more artificial muscles that is/are an integral part of the electrode array.

10. (canceled)

11. The method of claim 8, wherein:
the electrode array is a cochlear electrode array;
the action of inserting the implantable component into the recipient includes inserting the electrode array into a cochlea;
the first geometry is a curved geometry resulting at least in part from the curvature of the cochlea; and
the second geometry is a curved geometry having an average radius of curvature that is lower than that of the first geometry.

12. The method of claim 8, wherein:
the electrode array cochlear implant electrode array;
the action of inserting the implantable component into the recipient includes inserting the electrode array into a cochlea of a recipient in a third geometry;
the first geometry is a curved geometry resulting at least in part from the curvature of the cochlea; and
the third geometry is one of a substantially straight geometry or a negatively curved geometry relative to the curved geometry of the first geometry.

13. The method of claim 8, wherein:
the array is a cochlear implant electrode array;
the action of inserting the implantable component into the recipient includes inserting the electrode array into a cochlea of a recipient in the first geometry; and
the transformation from the first geometry to the second geometry takes less than 30 seconds.

14. (canceled)

15. The method of claim 8, wherein:
the electrode array has at least four actuators, and a carrier member of the electrode array is segmented to receive the four actuators in spaced apart manner with respective portions of the carrier member being located between at least two of the four actuators.

16. An implantable apparatus, comprising:
an electrode array including a plurality of electrodes and a carrier carrying electrodes, wherein the electrode array is configured so that, during and/or after insertion into a recipient, a local radius of curvatures at a first location and a second location more distal than the first location can be simultaneously controllably changed relative to one another.

17. The implantable apparatus of claim 16, wherein:
the local radius of curvature at the first location is controlled by one or more actuators located, relative to a longitudinal direction of the electrode array, between two electrodes.

18. The implantable apparatus of claim 16, wherein:
the local radius of curvature at the first location is controlled by relieving a tension force in the carrier, the relieving of tension increasing the local radius and/or establishing the local radius of curvature, respectively.

19. (canceled)

20. The implantable apparatus of claim 16, wherein:
the local radius of curvature at the first location is controlled by actuators in the carrier, wherein upon turning off the actuators, the local radius of curvature is increased and/or the local radius of curvature is established, respectively.

21. The implantable apparatus of claim 16, wherein:
the electrode array is configured so that, after insertion into a recipient, beyond that which results from tissue of the recipient causing such change and/or adoption, (i) a plurality of local radius of curvatures at a plurality of spatially separate locations, including the first location and the second location, can be controllably changed to be different from one another and/or (ii) the plurality of local radius of curvatures can be controllably obtained at the plurality of spatially separate locations from no radius of curvature at the respective plurality of spatially separate locations.

22. The implantable apparatus of claim 21, wherein:
the plurality of spatially separate locations include at least three.

23. (canceled)

24. The implantable apparatus of claim 16, wherein;
the local radius of curvature at the first location is controlled by actuators in the carrier, wherein upon turning on the actuators, the local radius of curvature is increased and/or the local radius of curvature is established, respectively.

25-26. (canceled)
